(19) United States
(12) Patent Application Publication
(10) Pub. No.: US 2022/0332754 A1
(43) Pub. Date: Oct. 20, 2022

(54) PEPTIDE SYNTHESIS

(71) Applicant: ORIGIN PEPTIDES LIMITED, Edinburgh (GB)

(72) Inventor: Sara TEN HAVE, Linlithgow Lothian (GB)

(73) Assignee: ORIGIN PEPTIDES LIMITED, Edinburgh (GB)

(21) Appl. No.: 17/309,150

(22) PCT Filed: Nov. 4, 2019
(86) PCT No.: PCT/GB2019/053123

§ 371 Date: Apr. 30, 2021

(30) Foreign Application Priority Data
Nov. 2, 2018 (GB) 1817932.5

(51) Int. Cl. C07K 1/02; C07K 14/62

(52) U.S. Cl. CPC C07K 1/02; C07K 14/62

(57) ABSTRACT

There is provided a method to synthesise peptides, a peptide being synthesised based on the amino acid sequence of a template peptide, peptides provided by the method and use of the peptides.

### FIELD

The present invention relates to a simple, cheap and environmentally safe method for synthesising peptides.

### BACKGROUND

A number of synthetic peptides are significant commercial or pharmaceutical products, ranging from the dipeptide sugar-substitute aspartame to clinically used hormones, such as oxytocin, adrenocorticotropic hormone, and calcitonin. Peptides are also increasingly being used in cosmetic applications for application to skin and hair, for example.

Peptides are currently generally made by recombinant means using bacterial or eukaryotic cell cultures or solid phase peptide synthesis, using FMOC or FBOC protected amino acids. Both processes are costly and purification of the peptides can be onerous. Moreover, for solid phase synthesis methods copious amounts of toxic organic solvents are required for the chemistry reactions and removal of the resulting waste material is approximately 10% of the overall cost of peptide production.

Native peptide ligation of peptides has been undertaken to provide proteins from large peptide fragments. Typically, this requires one half of the peptide to have a reactive thioester chemistry to enable the reaction. Further this technique relies on solid phase synthesis of two portions or halves of the desired peptide before these portions or halves are ligated together.

### SUMMARY

The present invention is based on studies concerning the synthesis of peptides without the use of complex chemistry, high temperature and/or pressure or DNA templates to facilitate peptide synthesis. Unlike solid phase synthesis of two portions or halves of the desired peptide and then ligation of these portions or halves together, the templated method described here, does not rely on having the majority of the peptide pre-synthesised, suitably only the constituent amino acids are required.

Unexpectedly, it has been found that a templated synthesis of peptides can occur by providing low amounts of energy to amino acids in solution.

For example, it was determined by the inventor that samples provided in the dark with a temperature differential of between 3-5° C. based around 21° C., were provided with sufficient energy to undertake the synthesis reaction, but that the reactions took longer than light exposed samples, and the long term results showed smaller structures generated.

Suitably, the templating method as used herein allows the use of any peptide, to make more of itself. Without wishing to be bound by theory, it is considered that providing a template (the peptide of choice to be synthesised) to the synthesis reaction imparts two benefits. Firstly, the cross-section of a peptide molecule, in a concentrated solution of amino acids, is greater than that of single amino acids. Sterically it is considered this means there will be more interactions with the template, and cause an accumulation or aggregation of the amino acids on the surface of the template peptide. Secondly the presence of the template peptide provides organisation of the amino acids by virtue of structural compatibility with like amino acids i.e. it is considered that arginine will ‘sit on’ an arginine in the template peptide due to the similar structure, likely retarding its movement. Suitably, it is considered that sequence selective molecular recognition on complementary surfaces has a pivotal role in peptide self replication. It is considered this will occur with the other amino acids in the sequence and bring them into close proximity to each other. The system provides energy sufficient enough to enable the peptide bond formation to occur between amino acids and for templated synthesis of the peptide to occur.

Advantageously, the present method is considered to be distinguished from prior art methods that require synthetic reactive chemistry on the termini of the molecules, be they peptides, DNA or RNA, or the pre-synthesis of the bulk of reacting peptide portions for ligation to each other.

According to a first aspect, there is provided a method of synthesising a peptide, the method comprising:
adding a quantity of a template peptide and amino acids capable of forming copies of the template peptide into an aqueous solution, and
providing a low amount of energy to the solution in order to synthesise copies of the template peptide in solution. Low can be defined as energy sufficient to overcome the required energy to form an amide bond. The energy may be provided by for example sunlight, for example the entire spectrum of light applied to the system including UV and part of the IR wavelength of the light spectrum.

Without wishing to be bound by theory, it has been suggested that in the absence of ionisation products, synthesis of peptide bonds would be favoured. Further, it has been proposed that formation of a di-peptide in an abiogenesis scenario is about 1.2 kcal/mol. It has also been proposed that this is 8× more difficult than adding an amino acid onto a peptide of any length, and 5× more difficult than joining 2 peptides of at least di-peptide size. Suitably the energy provided may be at least 0.15 kcal/mol, at least 0.24 kcal/mol, at least 0.3 kcal/mol, at least 0.6 kcal/mol, at least 1.2 kcal/mol. As would be understood to provide multiple peptide bonds sufficient energy can be provided. In embodiments energy provided can be the energy to allow 1 mol of di-peptide formation. In embodiments energy provided can be the energy to allow at least 2 mol of di-peptide formation, at least 3 mol of di-peptide formation, at least 4 mol of di-peptide formation, at least 5 mol of di-peptide formation, at least 10 mol of di-peptide formation, at least 15 mol of di-peptide formation, at least 50 mol of di-peptide formation or at least 100 mol of di-peptide formation. As will be appreciated, energy can be provided to provide the peptide formation required in accordance with the method.

To accurately discern a difference between energy that may be consumed in the dehydration reactions required to make peptide bonds the inventor used a controlled temperature environment and a control sample of PBS, and the PBS+amino acids solutions along with an ambient temperature measurement. Measurements were taken every 5 seconds for highly accurate heating and cooling curve description. The difference in area under the curve was determined to be 28962.97° C.²s, or 170° C.s, with the area being less in the PBS amino acids sample. The maximum of the peak in the PBS+amino acids was always less that the control, suggesting energy was always consumed in this system compared to the control. The resulting energy utilised by the PBS+amino acids sample was 1.31 kcal, suggesting that in one cycle of heating from 24° C. to 34° C. and cooling more than 1 mol of di-peptide formation was possible.

Suitably it is considered any form of energy may be provided as appropriate to allow bond formation, for example light, heat, or other electromagnetic radiation.

In particular, it has been observed that the provision of a low amount of energy (e.g. in the form of increased temperature and/or full spectrum light) to a solution of amino acids in the presence of a template peptide can promote the synthesis of copies of that peptide. Accordingly, the method can provide a simpler and more cost effective process of synthesising peptides, which does not require the use of nucleic acids, enzymes or co-enzymes, cells or cellular material and/or organic or environmentally harmful solvents. The method may be considered as an amplification process, wherein the amount of template peptide present in the solution is amplified as further copies of the template peptide are made.

Suitably the method of synthesising a peptide may occur in at least 2 minutes, at least 5 minutes, at least 10 minutes, at least 20 minutes, at least 30 minutes, at least 1 hour, at least 2 hours, at least 5 hours.

Suitably the synthesising of a peptide may occur in both light and dark conditions. The present invention is based on providing a low amount of energy to the aqueous solution. Previous wisdom has taught that large amounts of energy would be required in order to support chemical synthesis or conversion and so the low amounts of energy as used in accordance with the present method is unexpected. However, the experiments below clearly show production of peptides where there is no bacteria or other source of peptidase present and therefore provide proof that peptide production can occur based on a template reaction rather than an enzymatic one.

Without wishing to be bound by theory, the inventor considers that protein generation from the 20 standard amino acids only, in solution, for example utilizing energy input from the sun, would allow proteins to be the starting functional, structural and replicative material of life, which later recruited nucleic acids to more efficiently replicate themselves.

As would be understood by those of skill in the art, a large amount of energy may be provided to the system by means of application of extreme heat, intense microwave, or radioactive energy.

Mass spectrometry based proteomics has been used by the inventor as the primary mode of analysis. Given the peptide and protein generation is primarily stochastic, all spectra were initially identified using De Novo Sequencing (Peaks software), with a generated database being used to quantitatively analyse the data using MaxQuant. Structural characterization was carried out using Scanning Electron Microscopy (SEM) and Transmission Electron Microscopy (TEM). The quantification of the electron microscopy images was performed using Omero. Absence of DNA and RNA was tested using Fluorometric Quantitation (Qubit®, Life Technologies). Sterilisation of samples was performed using Gamma irradiation (1000 Gy), followed by standard peptide synthesis conditions, in hermetically sealed bags to additionally verify no contribution of living organisms to the peptide and protein synthesis.

The energy may be provided in the form of thermal energy or heat as provided by application of electromagnetic radiation, for example. This may include infrared, visible light and ultraviolet radiation, for example, or mixtures thereof. Alternatively, heat may be associated with the use of a heat pump, or simply due to the ambient or surrounding environment causing the temperature of the aqueous solution to rise.

In one embodiment the provision of energy may be constant. For example, the provision of energy may comprise exposing the solution to a constant source of full spectrum light for the duration of the peptide synthesis.

As used herein, “full spectrum light” may be taken to mean light covering the infrared to ultraviolet regions of the electromagnetic spectrum. For example, a full spectrum light source may provide light having wavelengths between about 300 nm to about 700 nm. The full spectrum light may be configured to mimic the composition of natural light.

The provision of energy may comprise maintaining the solution at a constant temperature. For example, the solution may be maintained at a temperature between about 10° C. and 100° C., between about 15° C. and 70° C., between about 20° C. and 50° C., or between about 30° C. and 45° C. By way of representative example, the solution may be maintained at a temperature about 35° C. or 40° C. Suitably the synthesising of a peptide may occur at room temperature, at least 21 degrees Celsius, at least 22 degrees Celsius, at least 23 degrees Celsius, at least 24 degrees Celsius, at least 25 degrees Celsius, at least 26 degrees Celsius, at least 27 degrees Celsius, at least 28 degrees Celsius, at least 29 degrees Celsius, at least 30 degrees Celsius, at least 31 degrees Celsius, at least 32 degrees Celsius, at least 33 degrees Celsius, at least 34 degrees Celsius, at least 35 degrees Celsius, at least 36 degrees Celsius, at least 37 degrees Celsius, at least 38 degrees Celsius, at least 39 degrees Celsius, at least 40 degrees Celsius.

In some cases, the solution may be maintained at a temperature of about 40° C. and exposed to a constant source of full spectrum light for the duration of the peptide synthesis.

Alternatively, the provision of energy to the aqueous solution may be carried out in a cyclical or repetitive manner, rather than in a constant manner. Thus, the energy may be provided for a period of time, and then stopped, before providing the energy again for a further period of time.

The periods of energy provision can be the same or different to the period when energy is not provided. For example, the energy, or light, may be provided for 2 hours, followed by a period without energy/light for a further two hours. Alternatively, the period of energy provision may be shorter or longer than the period without energy provision. In one embodiment the provision/non-provision of energy may be in the form of a diurnal rhythm. Thus, the provision/non-provision of energy follows a regular 24 hour cycle, as may be generally seen with the provision of light and darkness over a day.

In accordance with the above, the cyclical manner of providing energy to the solution may comprise periodically increasing the heat of the solution, for example by at least 0.25° C., 0.5° C., 1° C., 5° C., or even 10° C., 15° C., 20° C., or 100° C. Optionally the solution is permitted to cool to ambient temperature, before the application of heat occurs again. The cyclical manner of providing energy to the solution may correspond to the rise and fall in ambient temperature during a day/night cycle. This process may be repeated many, hundreds or even thousands of times.

In some cases, the solution may be exposed to a constant source of light (e.g. full spectrum light) whilst also being subjected to cyclical temperature oscillations as described above. Alternatively, the solution may be exposed to a cyclical provision of light (e.g. full spectrum light) under constant temperature conditions.

The method may be carried out in an aqueous solution. The solvent of the aqueous solution may comprise or consist essentially of pure or substantially pure water. For example, the aqueous solution may comprise or consist essentially of pure or substantially pure water, the template peptide and the amino acids capable of forming copies of the template peptide.

The aqueous solution may be sterile. As used herein, sterile may mean that the solution is free of biological contaminants. In some instances, the aqueous solution may be sterilised via the use of irradiation, such as gamma irradiation. Thus, the present method relates to abiotic peptide synthesis.

The aqueous solution may be free or substantially free from nucleic acids, enzymes, co-enzymes (such as adenosine triphosphate), cells, cellular material and/or organic solvents. For example, the aqueous solution may be free or substantially free from bacteria, viruses, eukaryotic cells and/or components thereof, such as organelles.

By way of example, pure or substantially pure water may be MilliQ® water, which is a form of ultrapure water of Type 1 (as defined by ISO 3696 (1987)). Other types of pure or substantially pure water may be provided by capacitive deionisation, reverse osmosis, carbon filtering, microfiltration, ultrafiltration, ultraviolet oxidation and the like.

Typically, any such pure or substantially pure water should have a low level of solids, low organics and low conductivity. For example, pure or substantially pure water may have less than 5 μg/ml solids, less than 1 μg/ml solids, or even less than 0.1 μg/ml solids. Additionally or alternatively, pure or substantially pure water may have an organics content of less than 100 μg/l, or less than 50 μg/l total organic carbon. Additionally or alternatively, pure or substantially pure water may have a conductivity of less than 1 μS·cm⁻¹, less than 0.1 μS·cm⁻¹, or even less than 0.01 μS·cm⁻¹ at 25° C.

Alternatively, the aqueous solution may comprise a source of phosphate. For example, the aqueous solution may comprise a source of phosphate other than ATP or ADP. An example of such a solution may be water comprising disodium hydrogen phosphate, or phosphate buffered saline solution. Phosphate buffered saline (PBS) may comprise disodium hydrogen phosphate, sodium chloride. In some formulations, phosphate buffered saline may further comprise potassium chloride and potassium dihydrogen phosphate. An exemplary PBS composition is shown below:

| Salt | Concentration (mmol/L) | Concentration (g/L) |
|---|---|---|
| NaCl | 137 | 8.0 |
| KCl | 2.7 | 0.2 |
| Na₂HPO₄ | 10 | 1.42 |
| KH₂PO₄ | 1.8 | 0.24 |

However, this should not be construed as limiting. Typically phosphate may be present in an amount of at least 50 μM, such as 100 μM, or 1 mM.

The use of a source of phosphate in the solution may be useful where a significant proportion of acidic amino acids are involved in the synthesis. By way of example, the presence of phosphate may assist in maintaining the template peptide (and any copies of the template peptide) in solution for the duration of the synthesis.

All available amino acids may be provided in the aqueous solution. For example, the aqueous solution may comprise a mixture of all of the proteinogenic amino acids. Alternatively, the amino acids provided to the solution may comprise a mixture of amino acids including (but not limited to) those amino acids present in the template peptide. By way of example, the solution may comprise a mixture of only those amino acids present in the template peptide (e.g. only those amino acids which are required to synthesise a desired peptide). The amino acids may be natural amino acids. That is, as found in nature. Suitably, the amino acids may not be activated amino acids. For example, the amino acids may not be linked to any coupling reagents such as carbonyl diimidazole.

The relative amounts of each amino acid provided or added to the solution may correspond to the relative amounts of each of the amino acids in the template peptide. For example, the amino acids may be provided in a molar ratio or stoichiometric amount which equates to the molar ratio or stoichiometric amount of each amino acid found in the template peptide (e.g. the peptide to be copied and synthesised). For example, if the peptide has the sequence, GlyAlaGly, Gly may be provided in the solution at double the concentration to Ala. In some cases, the stoichiometric amount of amino acids provided to the aqueous solution may be about equal to the stoichiometric amount of the amino acids found in the template peptide (e.g. within about ±10% or ±20% equal to the stoichiometric amount of the amino acids found in the template peptide). Alternatively, the amino acids may be provided in equimolar amounts.

The amino acid notations used herein are conventional and are as follows:

| Amino acid | Three letter code | One letter code |
|---|---|---|
| alanine | ala | A |
| arginine | arg | R |
| asparagine | asn | N |
| aspartic acid | asp | D |
| asparagine or aspartic acid | asx | B |
| cysteine | cys | C |
| glutamic acid | glu | E |
| glutamine | gln | Q |
| glutamine or glutamic acid | glx | Z |
| glycine | gly | G |
| histidine | his | H |
| isoleucine | ile | I |
| leucine | leu | L |
| lysine | lys | K |
| methionine | met | M |
| phenylalanine | phe | F |
| proline | pro | P |
| serine | ser | S |
| threonine | thr | T |
| tryptophan | try | W |
| tyrosine | tyr | Y |
| valine | val | V |

The present invention may be considered to extend to the proteinogenic amino acids, of which there a 20 standard genetically encoded (as identified above) and 3 additional amino acids—selenocysteine, pyrrolysine and N-formylmethionine. As well as more conventional L amino acids, the present invention may extend to the use of D amino acids, and post translationally modified amino acids such as phosphorylated, glycosylated and methylated residues.

The methods described herein may be applied to a wide variety of template peptides. The template peptide to be copied in the peptide synthesis may comprise any number of amino acids, such as between 2 and 200 amino acids. Suitably, the template peptide to be copied in the peptide synthesis may comprise at least 4 amino acids, at least 5 amino acids, at least 6 amino acids, at least 7 amino acids, at least 8 amino acids, at least 9 amino acids, at least 10 amino acids, at least 15 amino acids, at least 20 amino acids, at least 30 amino acids, at least 40 amino acids, at least 50 amino acids, at least 60 amino acids, at least 70 amino acids, at least 80 amino acids, at least 90 amino acids, at least 100 amino acids, at least 110 amino acids, at least 120 amino acids, at least 130 amino acids, at least 140 amino acids, at least 150 amino acids, at least 160 amino acids, at least 170 amino acids, at least 180 amino acids, at least 190 amino acids, at least 200 amino acids, at least 210 amino acids, at least 220 amino acids For example, it has been possible to synthesise copies of relatively long and/or challenging peptide sequences, using the methods described herein. The methods described herein may be effective for the templated synthesis of peptides having a particular structural conformation, such as a conformation determined by one or more disulfide bonds between amino acids in the peptide. Generally, the synthesis of a peptide with a particular structural conformation may only be expected to occur in the presence of a molecular chaperone (e.g. a protein that can assist in the folding and assembly of a peptide into a particular structural conformation). However, it has surprisingly been found that the methods described herein can be used to synthesise copies of such peptides. For example, it has been possible to synthesise insulin (a relatively long peptide having three disulfide bonds and a particular structural conformation) using the methods described herein. The template peptide may not be an amyloid peptide.

Suitably the template peptide may have a secondary structure. Suitably the secondary structure may be substantially helical. Suitably the secondary structure may be substantially beta sheet. Suitably the secondary structure of the template structure may be a combination of helical and beta sheet.

The quantity of template peptide may be added to the aqueous solution to provide a concentration of the template peptide in the solution. Typically, it is possible to synthesise copies of the template peptide in solution using even very low amounts of the template peptide. Therefore, the template peptide may be provided in any concentration. The concentration of the template peptide in solution may also vary in relation to the length of the peptide. Higher concentrations of template peptide may be used to increase the rate of synthesis of copies of the template peptide.

It is considered that utilising less template in the starting material will result in a longer synthesis time. By increasing the template present then the reaction may proceed more quickly.

The relative quantities of amino acids and template peptide added to the aqueous solution may also be varied. For example, the total weight of all of the amino acids and the weight of the template peptide may be provided in a w/w (weight by weight) ratio of anywhere between 20,000 to 1 and 10 to 1. For example, a w/w ratio between 15,000 to 1; 10,000 to 1; 5 000 to 1; 1 000 to 1; 100 to 1 and 10 to 1 w/w; By way of further example, the solution may comprise about 21.5 to 1 w/w of the amino acids to the template peptide.

The total weight of the amino acids may be provided in such an amount to provide a solution having a concentration between about 0.001 g/mL and 10 g/mL, or between about 0.005 g/mL and 5 g/mL, or between about 0.01 g/mL and 1 g/mL. For example, the solution may have a concentration of amino acids about 0.02 g/mL. In some cases, it should be appreciated that the concentration of the solution may be dependent upon the composition of the template peptide. For example, different amino acids have different water solubilities and so where a template peptide comprises a relatively higher proportion of amino acids having higher water solubility, a more concentrated solution may be used in the peptide synthesis.

The amino acids and template peptide may be added to the aqueous solution sequentially or simultaneously. The method may facilitate a peptide synthesis, wherein all the reagents required (e.g. the amino acids and template peptide) may be added into the solution at the outset of the synthesis. Consequently, the methods described herein may obviate the need to use complex chemistries and/or multistep processes (such as the need to use protecting group strategies and/or solid phase components).

By providing a template peptide in the method, it can be possible to obtain copies of the template peptide in high quantity and quality. Typically, at least 80%, 90%, 95%, 99%, 99.5% or even higher amounts of the peptides produced are identical to the template peptide.

The progress of the peptide synthesis may be monitored. For example, the amount of synthesised peptide in the aqueous solution may be monitored using mass-spectrometry (MS) techniques (e.g. matrix-assisted laser desorption/ionization time-of-flight mass spectrometry (MALDI-TOF-MS) or ion-trap electrospray MS).

As will be appreciated, an increase in the amount of synthesised peptide may be observed initially, as the amino acids react to form copies of the template peptide. The amount of synthesised peptide may then plateau (e.g. as all the starting amino acids are consumed). Alternatively, after an initial increase, the amount of synthesised peptide may fall (e.g. if additional amino acids start to add to the synthesised peptide, increasing the length of the peptide chain). Therefore, in at least some cases the peptide synthesis may be terminated when an observed amount of synthesised peptide plateaus or decreases.

The duration of the peptide synthesis may be dependent upon the length of the template peptide and/or the nature of the amino acids within it. For example, shorter template peptides may be copied more rapidly than longer template peptides. Therefore, monitoring the peptide synthesis can be used to experimentally determine a duration of the process that will provide sufficient quantities of the desired peptide. By way of example, the synthesis process may take place over a period of hours (such as 6, 12, 18 or 24 hours) or days to provide sufficient quantities of the desired peptide. For example, the synthesis process may take place over at least 1, 2, 3, 4, or 5 days, or even up to 12, 20, 25, 30 or 50 or more days.

Termination of the peptide synthesis may be carried out by removing the low amount of energy from the solution. By way of representative example, the peptide synthesis may be terminated by cooling the aqueous solution (e.g. to a temperature below 10° C., below about 5° C. or below about 0° C. Alternatively or additionally, peptide synthesis may be finally terminated by separation of the synthesised peptide from the aqueous solution.

The peptide synthesised in the method may require minimal purification (in some cases no purification). For example, the peptide synthesised in the method may be reasonably homogeneous.

If further purification is needed, the synthesised peptide may be purified from the aqueous solution in which the synthesis process takes place, in order to remove desired synthesised peptides from the amino acids and shorter, longer and/or miscopied peptides as necessary.

Typically purification may be carried out by chromatographic means, such as high-performance liquid chromatography (HPLC). Examples of suitable HPLC methods include reversed-phase HPLC, ion exchange HPLC and gel-filtration HPLC and such HPLC methods may be carried out individually or in combination/tandem. The progress of peptide purification may be monitored, such as by mass-spectrometry (MS) techniques, including matrix-assisted laser desorption/ionization time-of-flight mass spectrometry (MALDI-TOF-MS) or ion-trap electrospray MS. Sequencing by Edman degradation sequence analysis, or in tandem with MS, for example, can be used to confirm peptide homogeneity. Suitably purification may occur using binding members which can selectively bind the peptide/protein of interest provided by the method, for example an antibody or fragment thereof. Suitably the peptide/protein of interest provided by the method may be provided with a tag to allow it to be purified. Suitably, beads with a binding member specific for the peptide/protein of interest provided by the method or to a tag attached to the/protein of interest provided by the method may be utilised.

The method of synthesising the peptide may be carried out at atmospheric pressure (e.g. 1 bar ±0.2). Additionally, or alternatively, the method may be carried out in air, e.g. in the presence of oxygen. For example, typically the method does not require a reducing gas environment and/or high pressures. The templated synthesis of peptides may occur without the application of significant amounts of energy (e.g. as would be provided for example by an electric spark or the like).

The method may be a batch process, or the process may be a continuous one, with synthesised peptides removed over time.

The method may be carried out in a receptacle or reaction vessel. In some instances, the receptacle or reaction vessel may be covered or sealed (e.g. to reduce the risk of contamination).

According to a further aspect of the invention, there is provided a use of a template peptide for amplification of itself. That is, use of a peptide as a template for peptide amplification. The amplification comprises duplication of the peptide using the peptide as a template.

For example, the amplification may comprise the synthesis of further copies of the template peptide. The amplification may be facilitated by the template peptide itself in the absence of nucleic acids, enzymes or co-enzymes, cells or cellular material. That is, the amplification does not require enzymes capable of forming peptide bonds, e.g. peptidyl transferases. The amplification solution does require amino acids for incorporation into the growing duplicated peptide strand.

Moreover, throughout this specification the term “comprising” is used to denote that embodiments of the invention “comprise” the noted features and as such, may also include other features. However, in the context of this invention, the term “comprising” may also encompass embodiments in which the invention “consists essentially of” the relevant features or “consists of” the relevant features.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

### DETAILED DESCRIPTION OF THE INVENTION

The present invention will now be further described by way of example and with reference to the following figures which show:

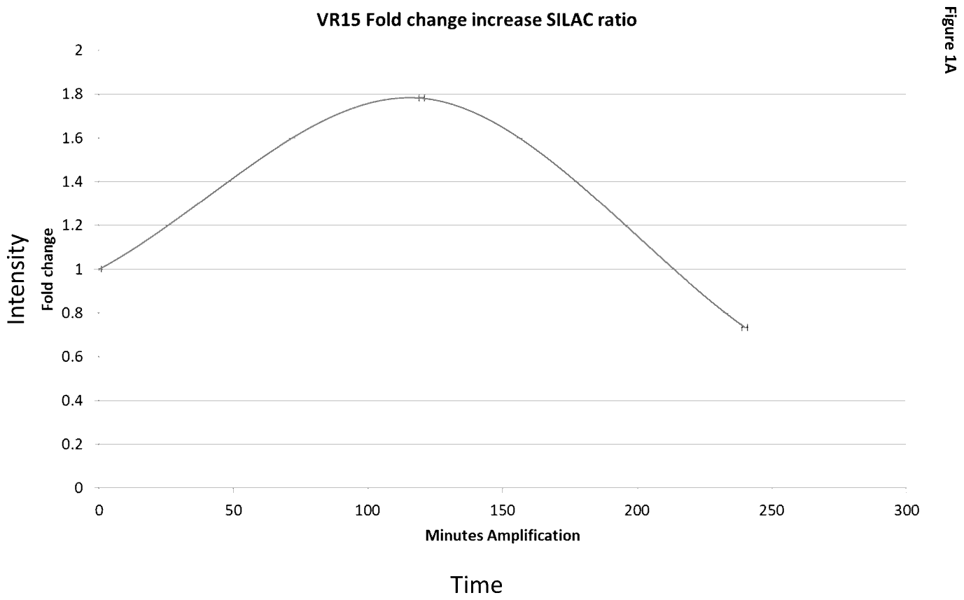

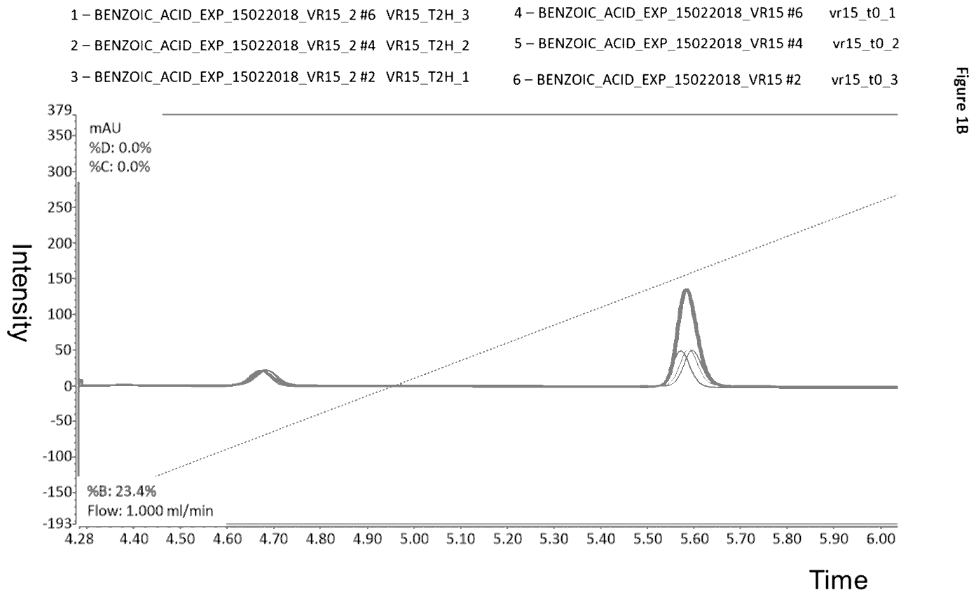

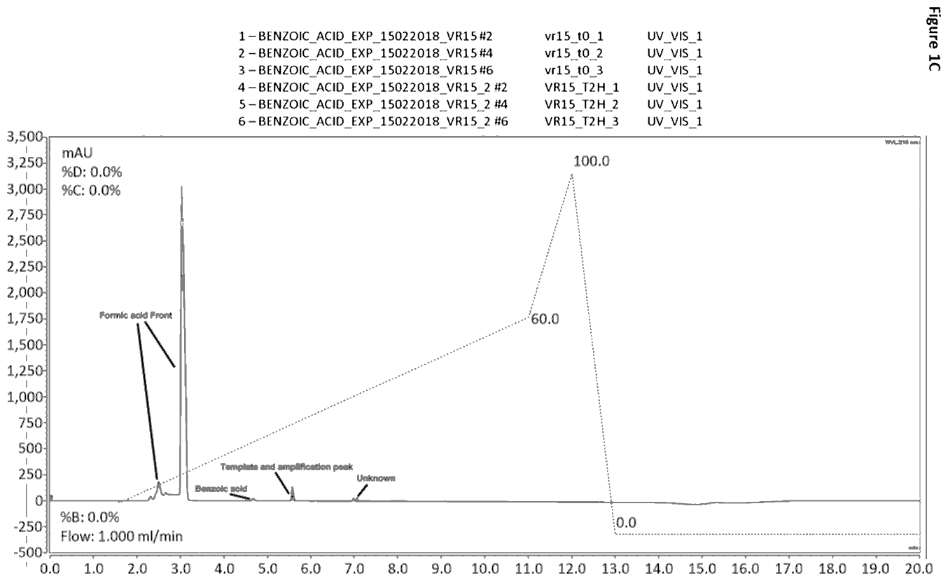

FIG. 1A shows the quantitative analysis of a tem plated peptide synthesis using mass spectrometry (Thermo Q Exactive Orbitrap) for a synthesised peptide VR15 (SEQ ID NO: 1—VPDNLQQSLSDEAQR—this peptide does not occur in nature). The newly synthesised peptides are differentiated from the template peptide by the incorporation of heavy Arginine (R, denoted SILAC here), which means this peptide can be measured distinctly from its templating peptide in Mass Spectrometric analysis. B shows the UV absorbance of the samples over time, with the significant increase in signal of the new peptide compared to time point 0. C shows the complete chromatogram. All samples were amplified with an internal standard (benzoic acid) which does not change throughout the process—this is seen in B and C. D shows the intensity as measured in the Mass Spectrometer (intensity represents ions detected within the mass spectrometer and is a direct indication of quantity).

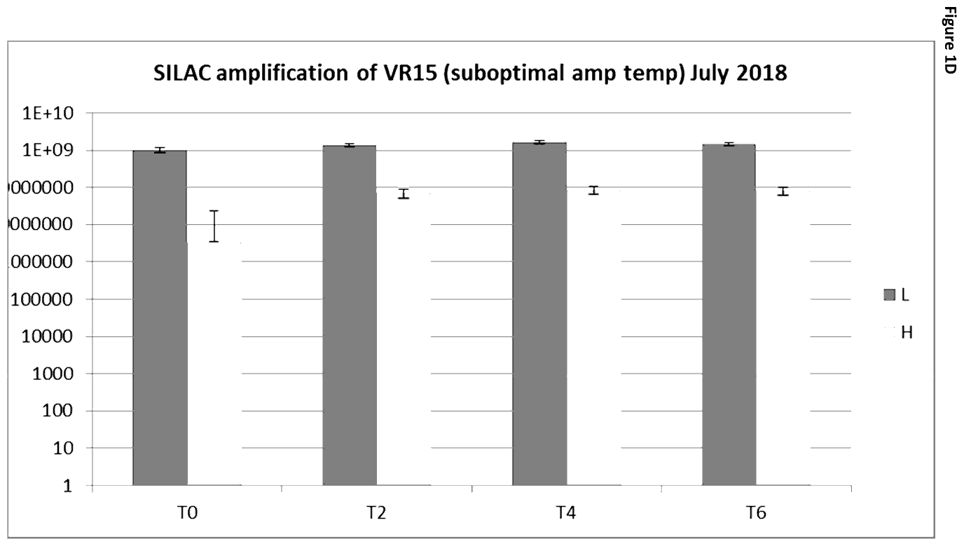

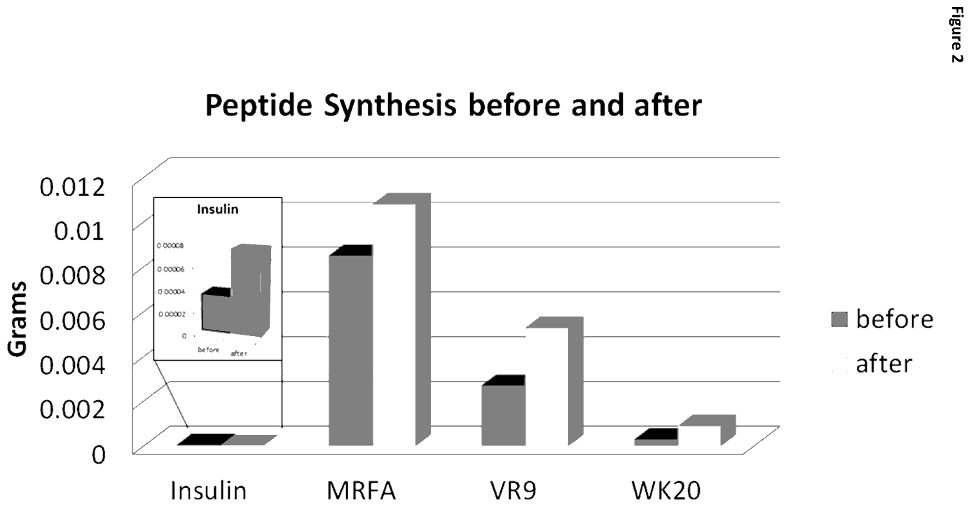

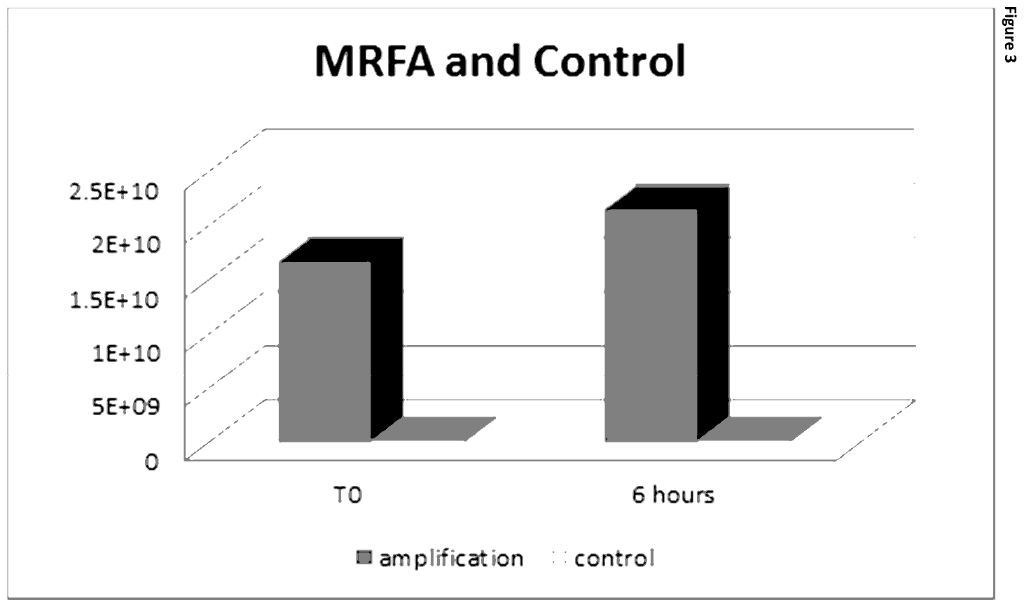

FIG. 2 shows the amount by weight of the template peptide before and after the templated peptide synthesis, of four different peptides; Insulin (110 amino acids long), MRFA (4 amino acids long), VR9 (synthetic peptide not occurring in nature VMDSSYLSR, 9 amino acids long SEQ ID NO: 2), and WK20 SEQ ID NO: 3 (synthetic peptide not occurring in nature, WRWLEHNVVEGNAVNLMFSK). This shows that peptides of any length can be amplified, with structure and disulphide bonds not presenting an issue in the amplification process.

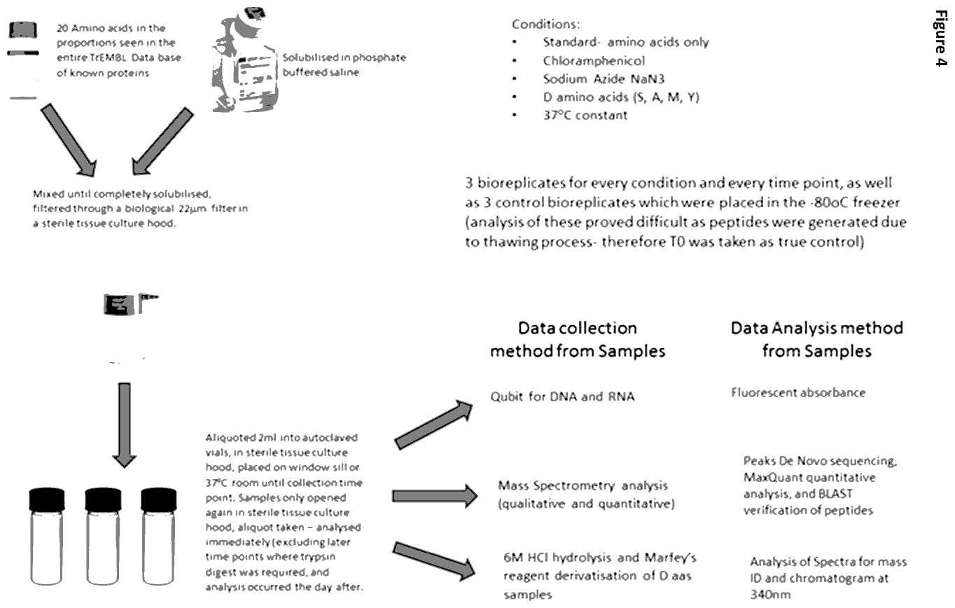

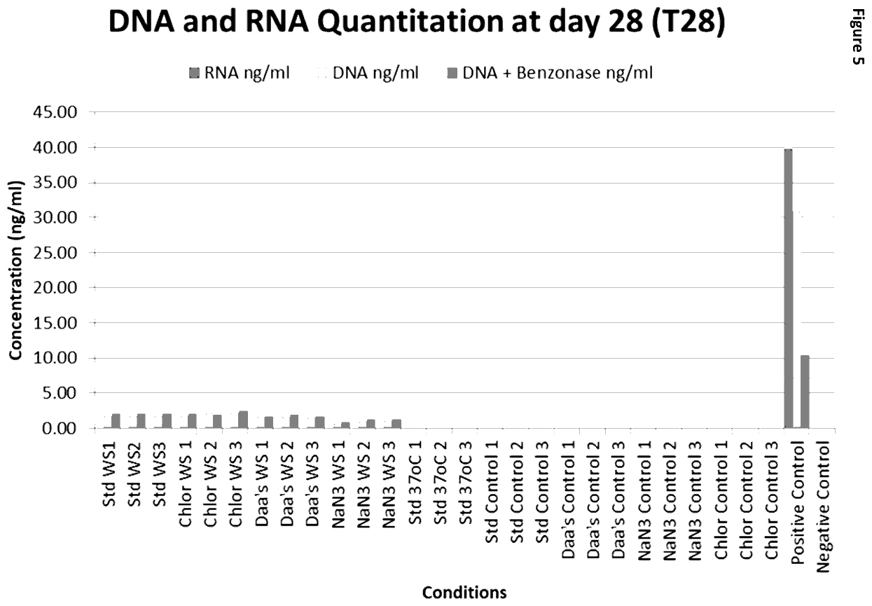

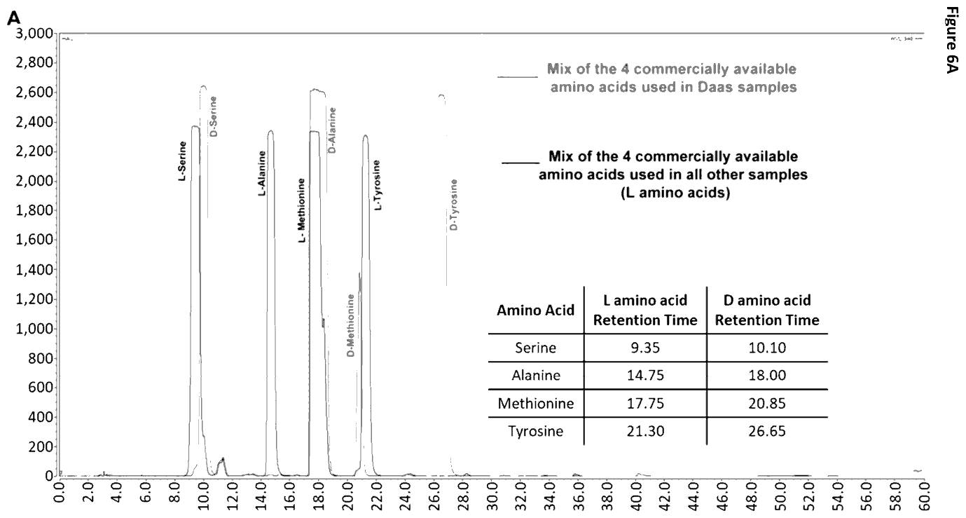

FIG. 3 shows the quantitative analysis of MRFA peptide synthesis using mass spectrometry (Thermo Q Exactive Orbitrap) in the presence of a MRFA template peptide (“amplification”) and in the absence of a template peptide (“control”). This shows that the addition of the template does indeed catalyse production of the peptide.

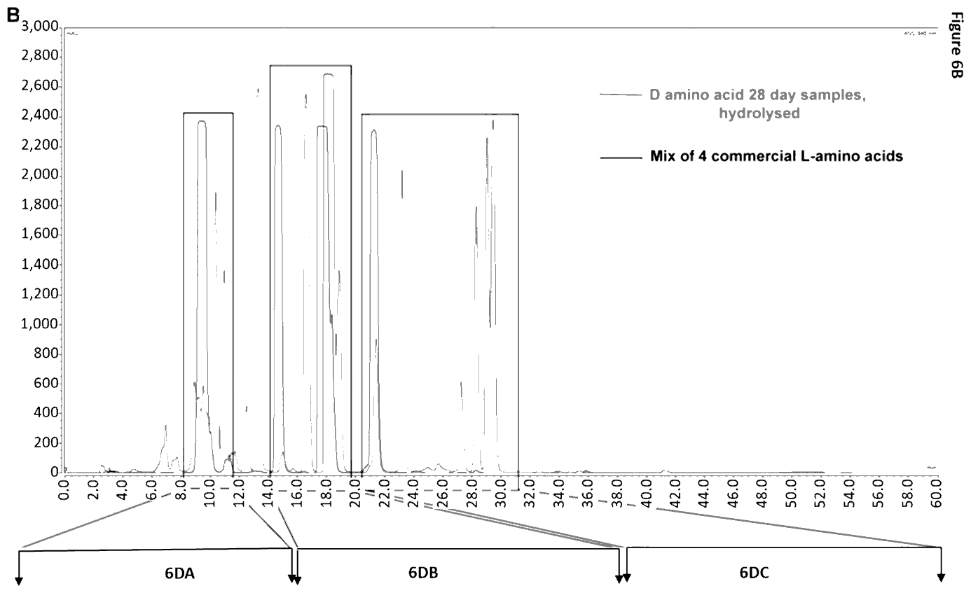

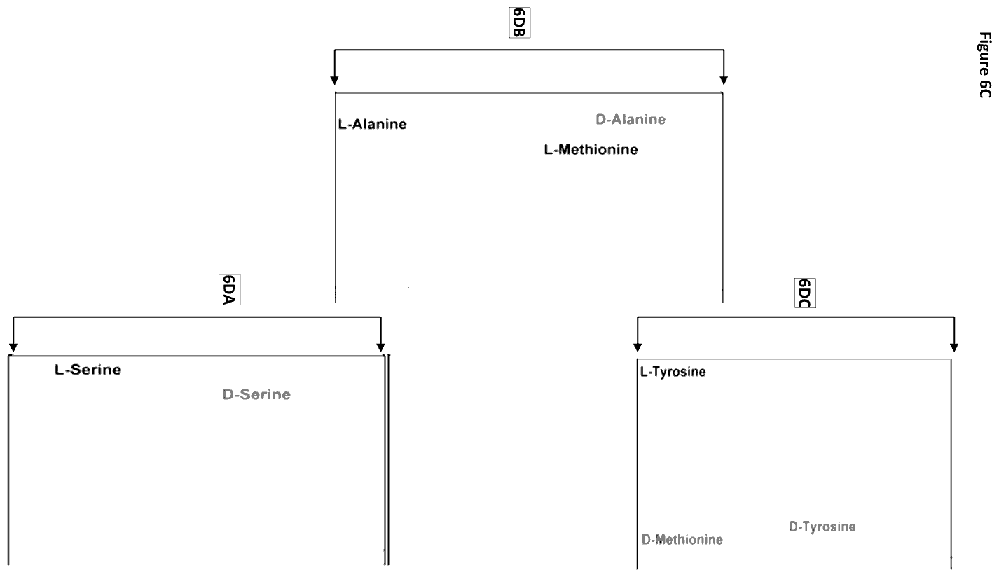

FIG. 4 shows the experimental designs for the subsequent experiments in order to demonstrate the lack of contamination present in the exemplified peptide synthesis process. The inclusion of additional control samples with sodium azide (NaN₃), chloramphenicol (Chlor) and D amino acids (Daa's) were used to further guarantee the absence of biological contamination.

FIG. 5 shows the results of the testing for presence of DNA and RNA. The samples were tested for the presence of DNA and RNA using the high sensitivity Qubit® kits and representations of the observed intensities are shown. No RNA was ever detected and DNA was detected after the samples changed colour. To verify whether this was in fact DNA, benzonase (which digests DNA, and would therefore eliminate the detectable signal of the DNA in the samples) was applied to the samples and left to incubate. A positive control was always used and the intensity of the positive control always reduced by at least half, while the peptide synthesis samples “DNA intensity” was never reduced significantly. This verifies that there is not “life” present in the peptide synthesis samples (i.e. DNA and RNA).

FIG. 6 shows the results of the Marfey's Reagent derivatisation of D and L amino acids A. This chromatogram shows the overlay of a mixture of 4 commercial L amino acids (the black trace) and a mixture of the same 4 amino acids but the D isomers (the red trace). The delay in retention time can be seen in the D amino acids due to their derivatisation with Marfey's Reagent (FDAA, 1-fluoro-2-4-dinitrophenyl-5-L-alanine amide). B shows the abio D amino acids sample taken at day 28, hydrolysed into single amino acids and derivatised with Marfey's (red trace) overlaid with the 4 commercial L amino acid chromatogram (black trace). It was observed that there was no conversion of D amino acids into L amino acids (which may be expected to occur if a biological life form contaminating the sample were utilising the D amino acids).

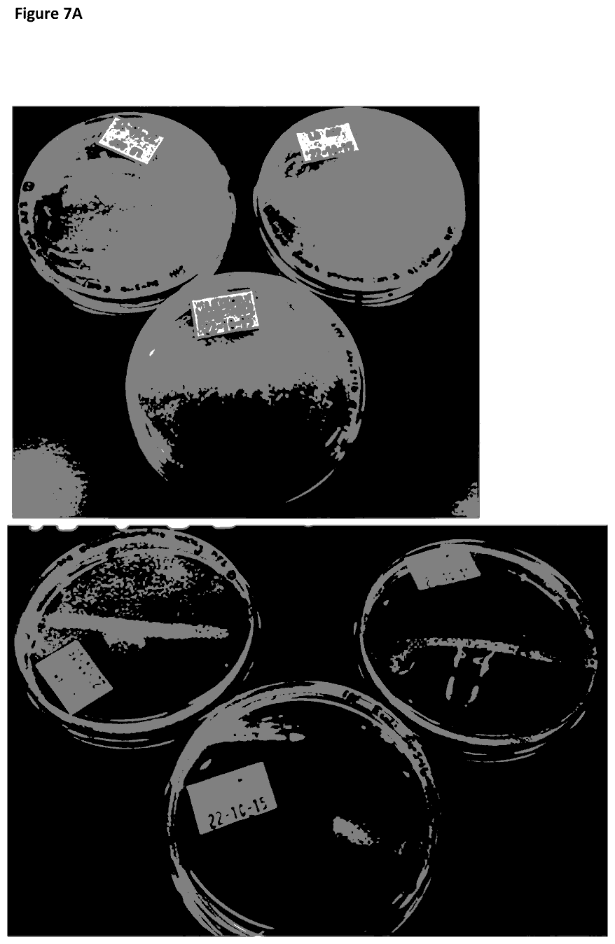

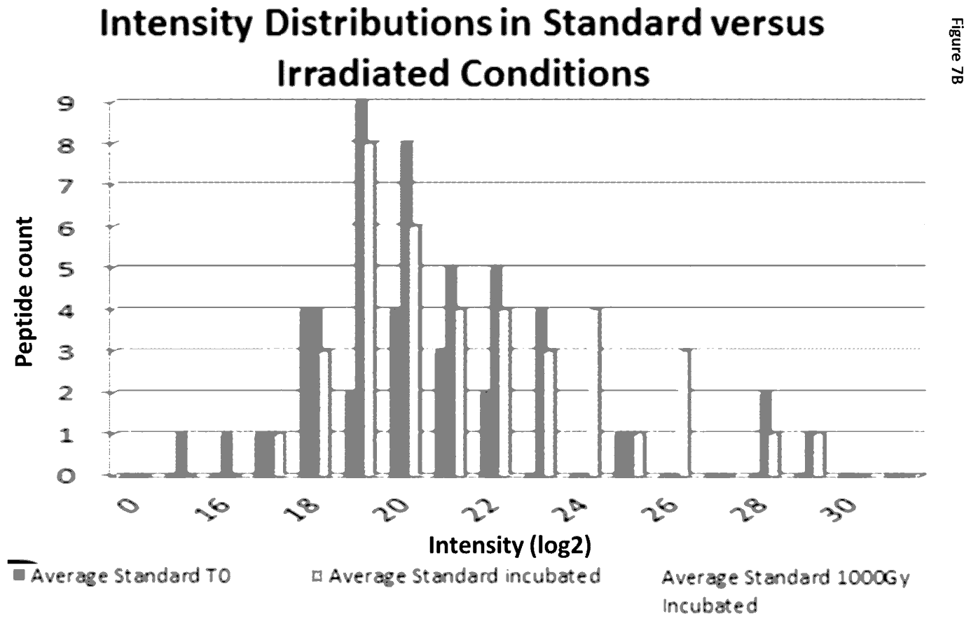

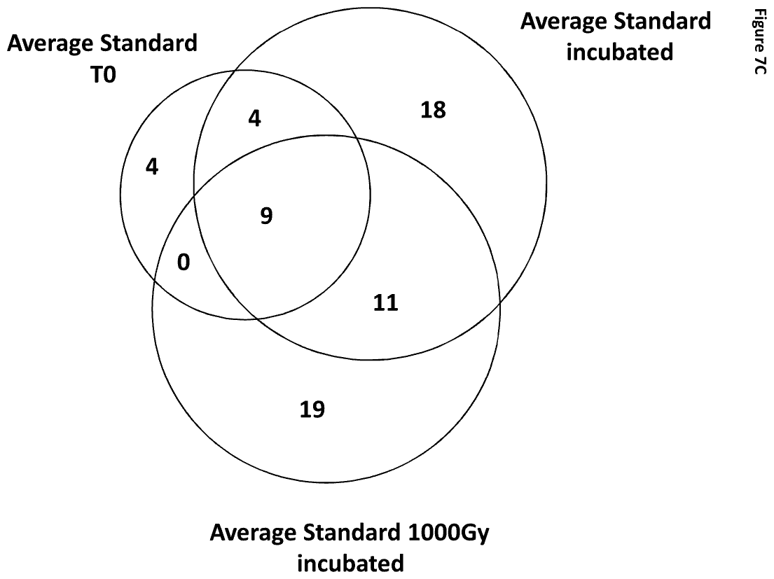

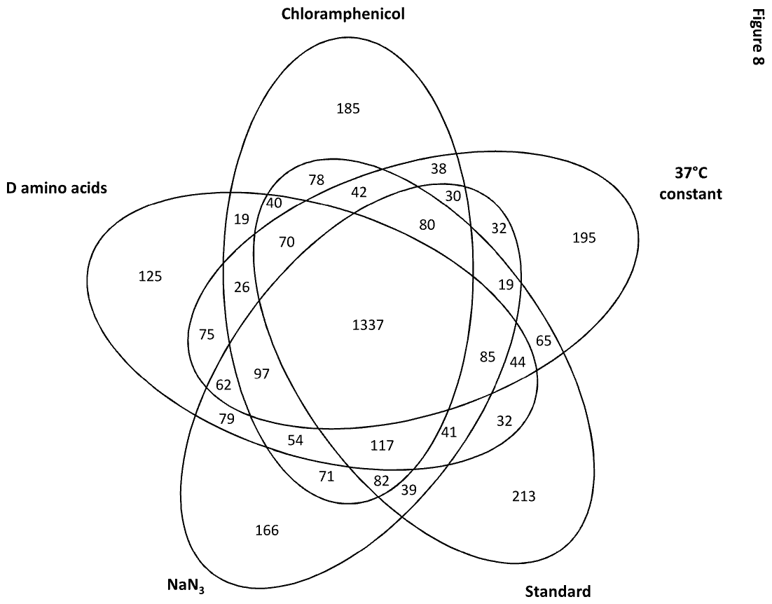

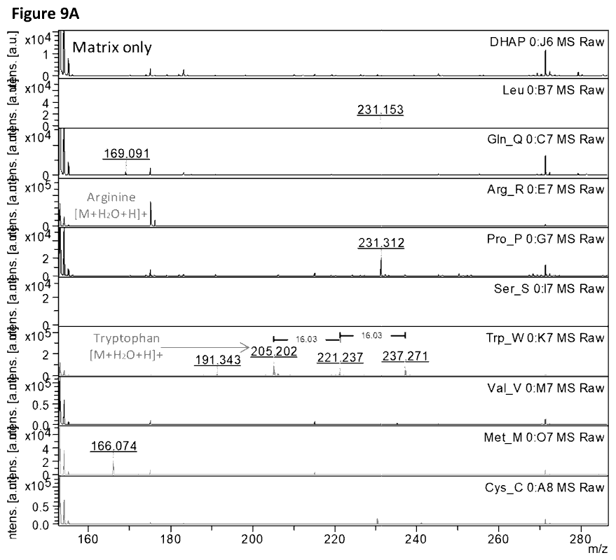

FIG. 7 shows the results of the gamma irradiation experiment (all samples run in triplicate). A positive control for sterilization was included in the form of an E. coli culture. The non-irradiated samples (A left side) show numerous colonies, and the irradiated samples (A right side) show after 1000 Gy irradiation no growth at all, demonstrating effective sterilisation. B is a histogram describing the peptide intensity distributions. The dark green population is the starting material, which has a low peptide count (as this was a template guided experiment—meaning the only products were the peptide itself and a small percentage of miscopies—which occur with this process) as well as lower median intensity. The standard incubated samples generated more peptides of higher intensity (i.e. more copies of peptides), with the irradiated samples showing a similar increase in peptide generation to that of the non-irradiated sample. The lack of distinct difference between these two sample sets suggests this is a chemical process not influenced by external living contamination of any sort. C is a Venn diagram showing the co-occurrence of peptides generated between conditions. The templated generation of peptides between the irradiated and not irradiated samples are equivalent, demonstrating the reaction is a chemical one, independent of bacterial/biological contamination.

FIG. 8 shows a Venn diagram showing the co-occurrence of detected peptides between various conditions (chloramphenicol, 37° C., standard, sodium azide (NaN₃), and D Amino Acids). Chloramphenicol (broad spectrum antibiotic), sodium azide (gram negative bacterioside) and D amino acids (most living organisms require L-amino acids—and if D-amino acids are utilised by a living organism they will be converted into L-amino acids—which didn't occur in this case (see Marfey's reagent experiment) in the concentrations used in these experiments should eliminate bacterial growth. Peptide synthesis in all experiments is comparable and shows the chemistry basis for the synthesis as opposed to biological origin of the peptides.

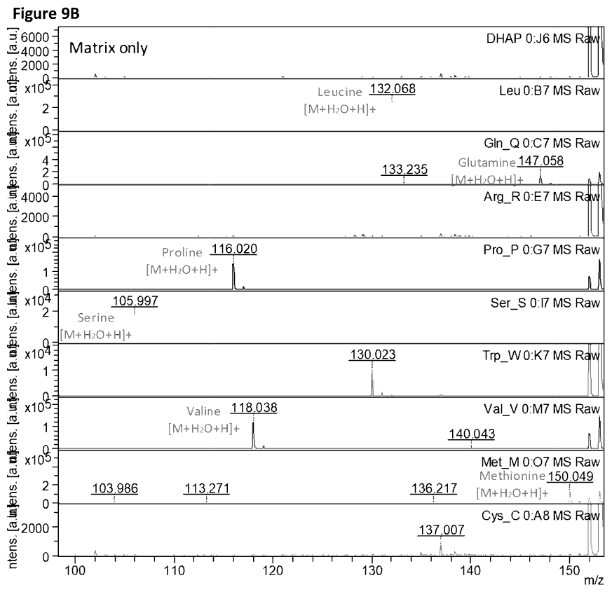

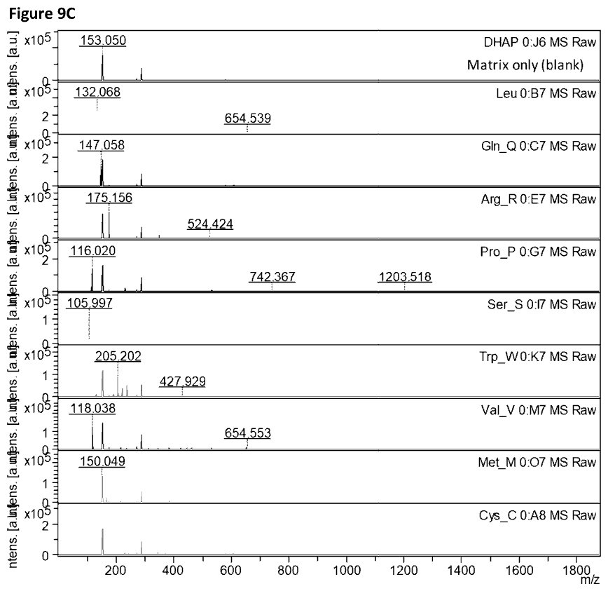

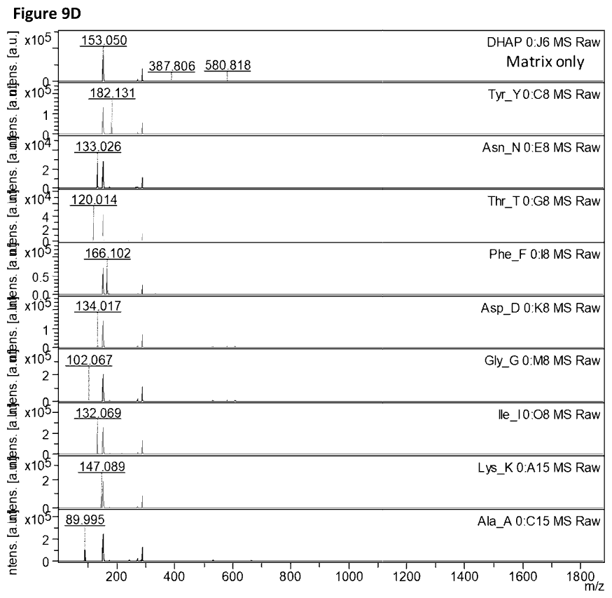

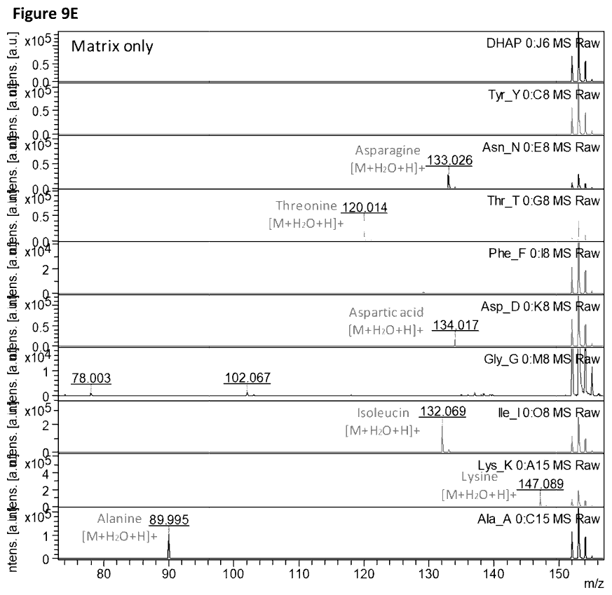

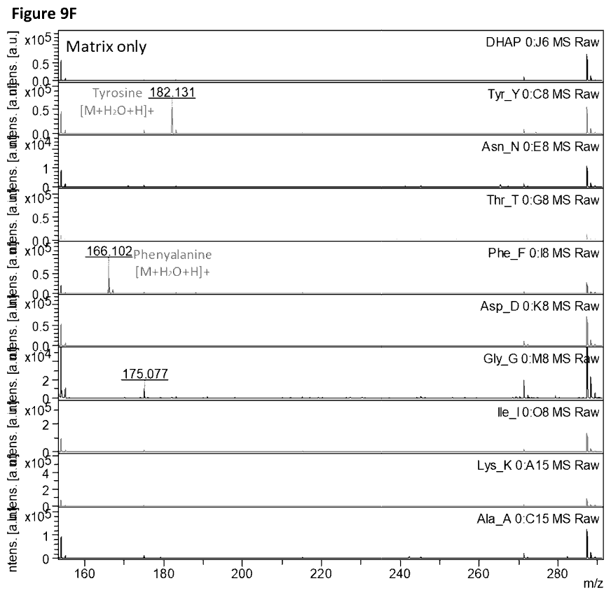

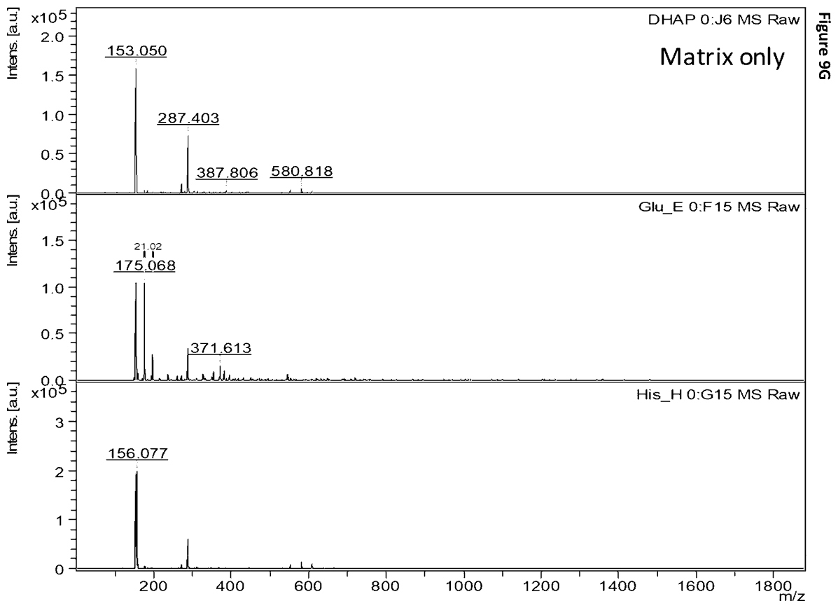

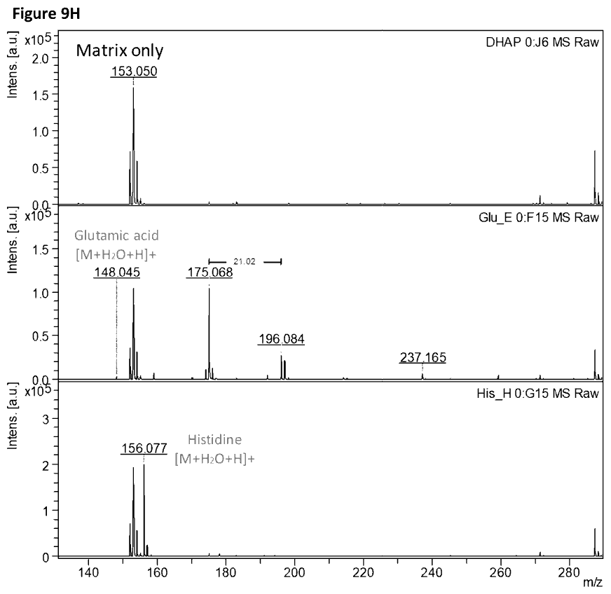

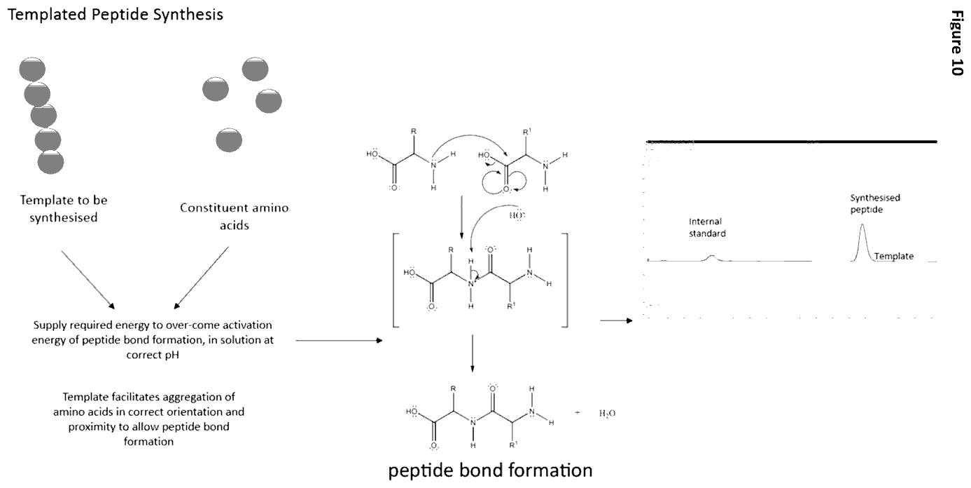

FIG. 9 shows MALDI-TOF Mass Spectrometry of Commercial Amino Acids. These spectra show the starting materials are pure and free from any larger molecules which could contribute to the peptides we see in the experiments. A. MALDI-TOF spectra of leucine, glutamine, arginine, proline, serine, tryptophan, valine, methionine and cysteine; B. MALDI-TOF zoomed mass spectra of leucine, glutamine, arginine, proline, serine, tryptophan, valine, methionine and cysteine; C. MALDI-TOF zoomed mass spectra of leucine, glutamine, arginine, proline, serine, tryptophan, valine, methionine and cysteine; D. MALDI-TOF mass spectra of tyrosine, asparagine, threonine, phenylalanine, aspartic acid, glycine, isoleucine, lysine, and alanine; E. MALDI-TOF zoomed mass spectra of tyrosine, asparagine, threonine, phenylalanine, aspartic acid, glycine, isoleucine, lysine, and alanine; F. MALDI-TOF zoomed mass spectra of tyrosine, asparagine, threonine, phenylalanine, aspartic acid, glycine, isoleucine, lysine, and alanine; G. MALDI-TOF mass spectra of glutamic acid and histidine; and H. MALDI-TOF zoomed mass spectra of glutamic acid and histidine.

FIG. 10 illustrates a schematic of the method of the present invention

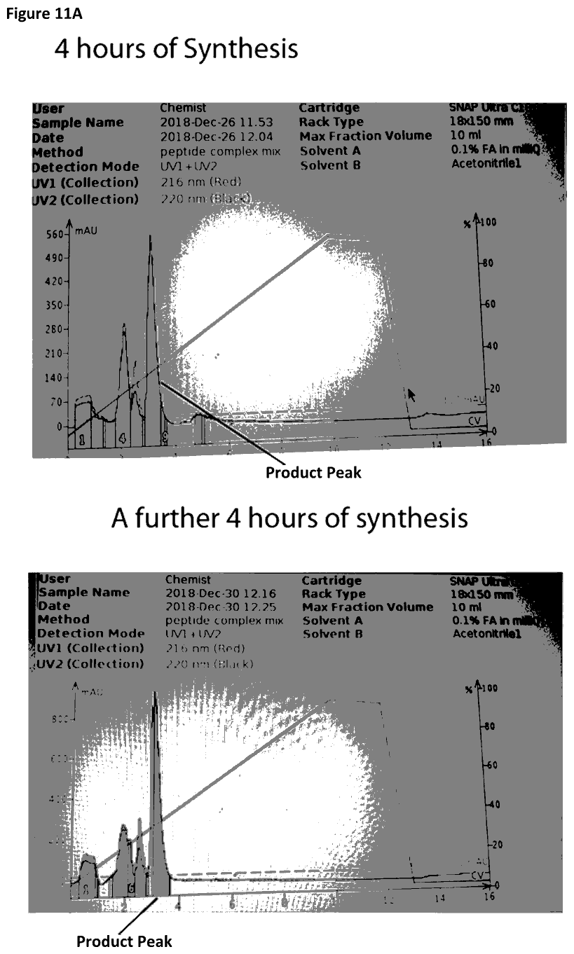

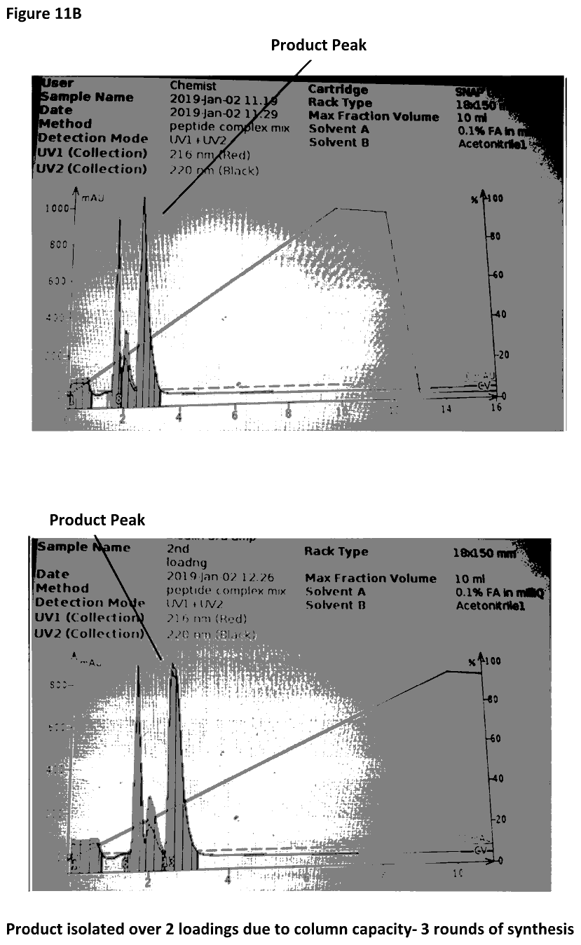

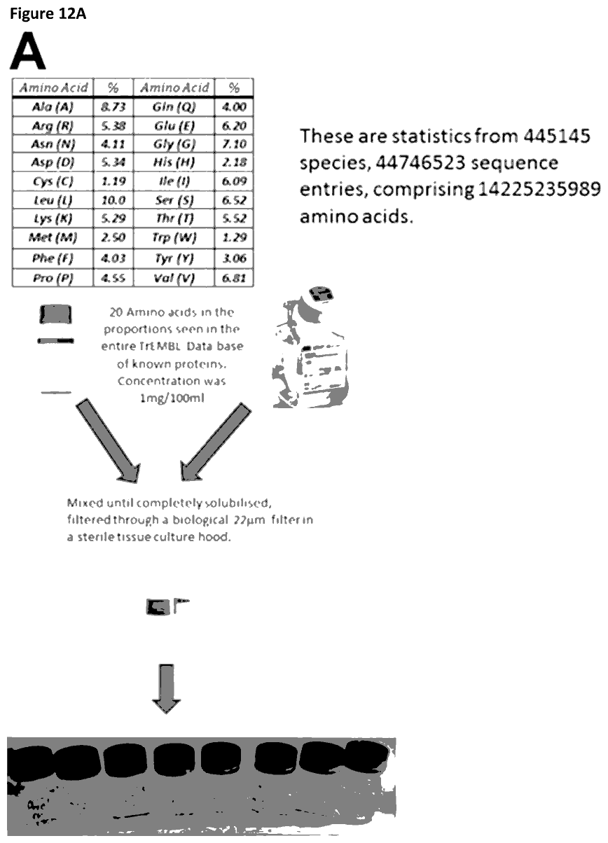

FIG. 11 illustrates insulin synthesis using the method of the present invention.

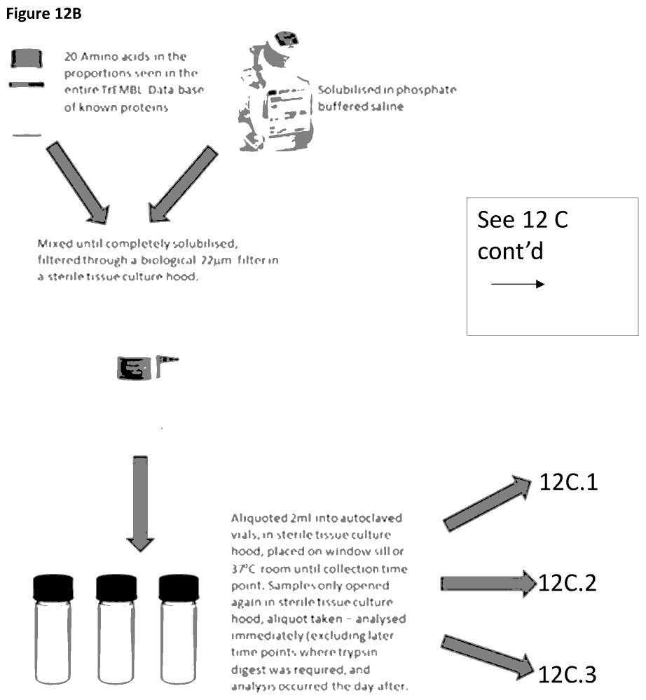

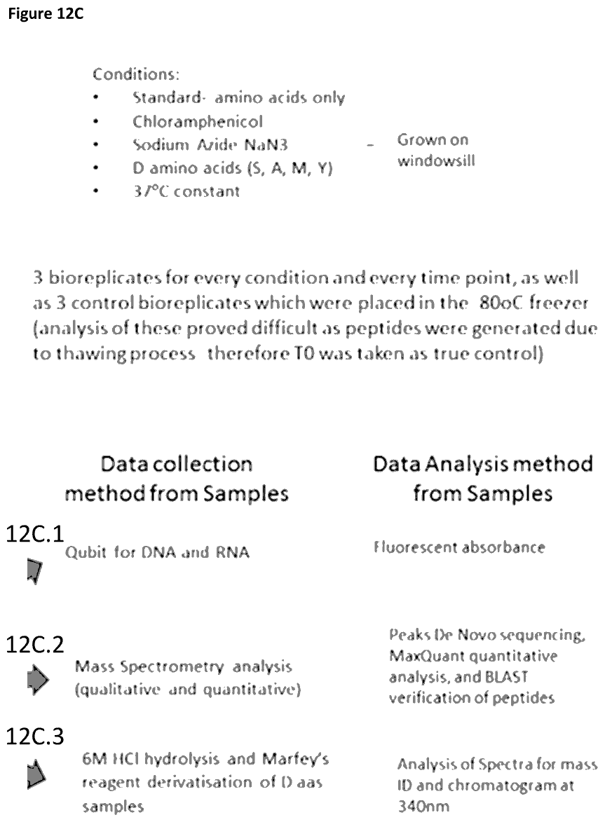

FIG. 12 (A) illustrates an experimental set up to test the necessity of sun light and phosphate to the spontaneous polymerization of amino acids into peptides and proteins. All samples were run in triplicate (B) illustrates the experimental set up of all subsequent experiments with greater sterility. The inclusion of additional control samples with sodium azide, chloramphenicol and D amino acids were used to further guarantee the absence of biological contamination

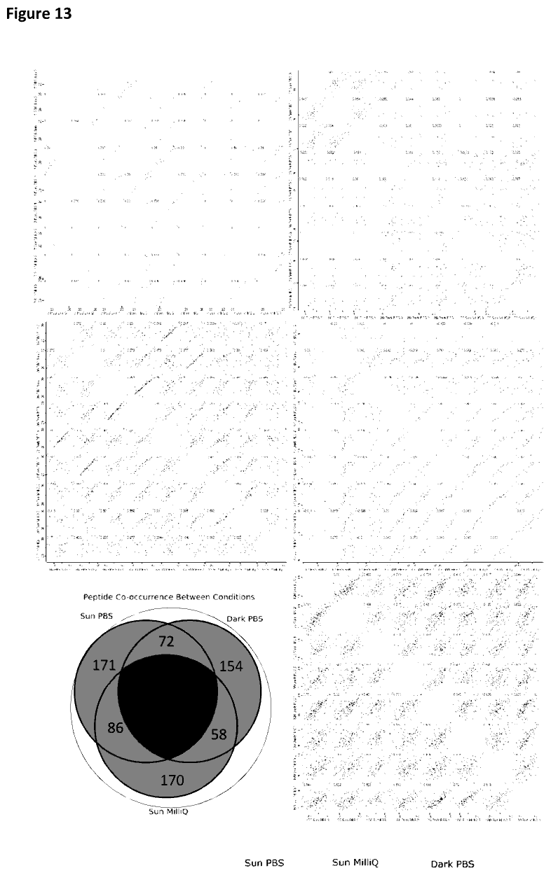

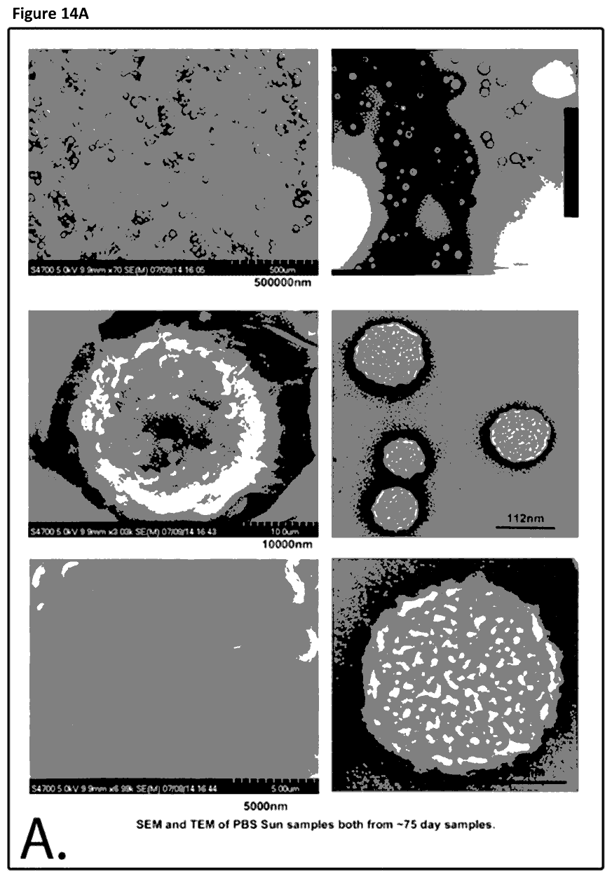

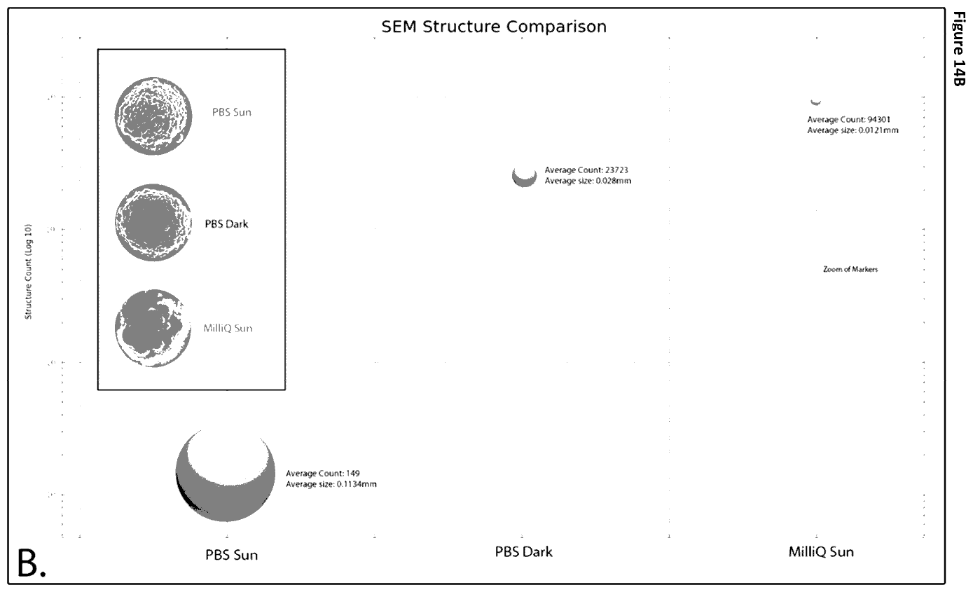

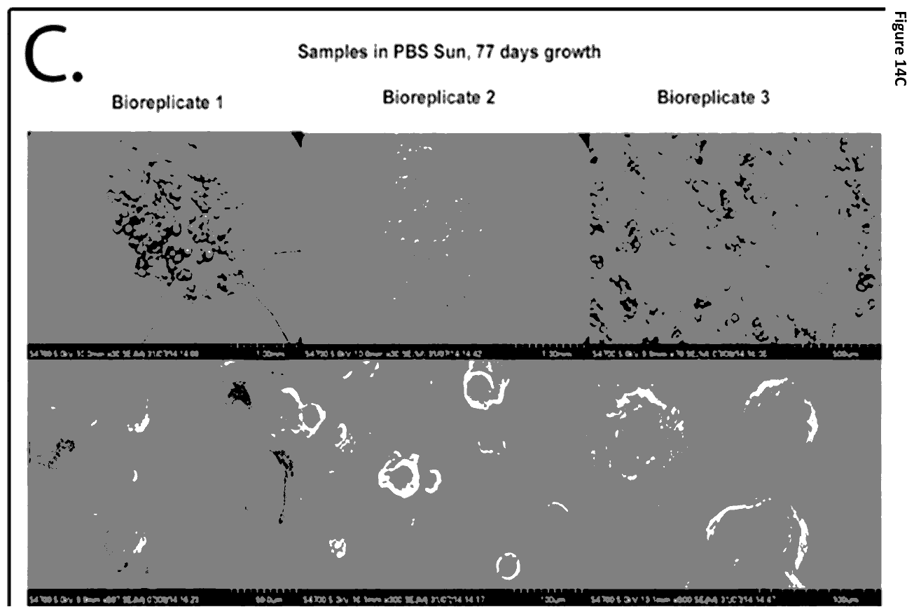

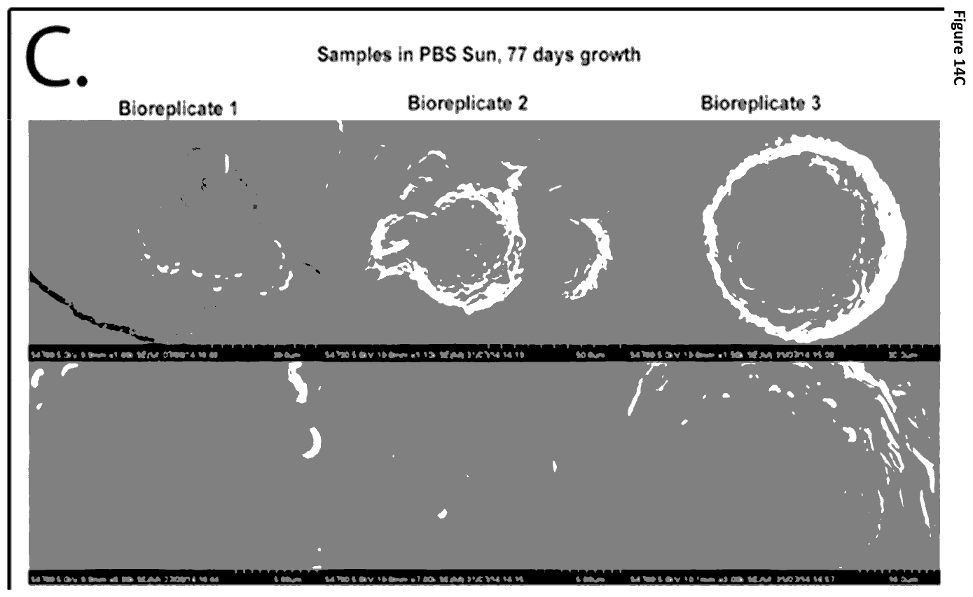

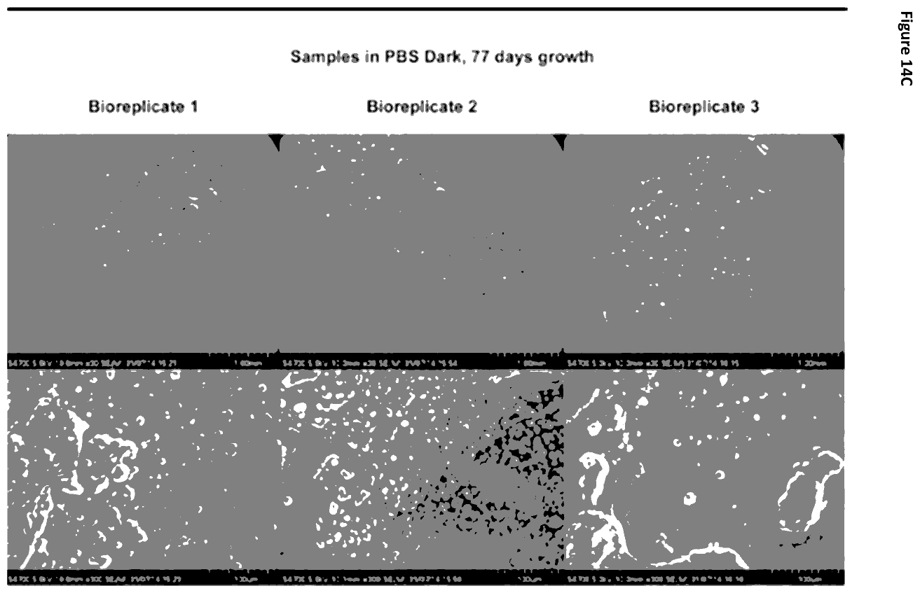

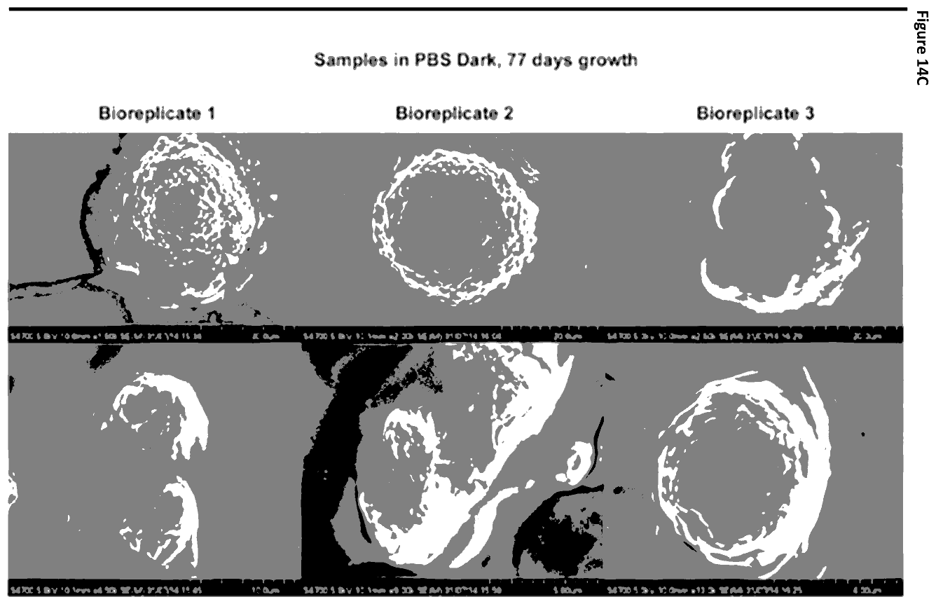

FIG. 13 illustrates correlations (Log2 of peptide intensities) of all collected time points in the experiment of FIG. 12 and the venn diagram shows the majority of peptides seen were shared between samples.

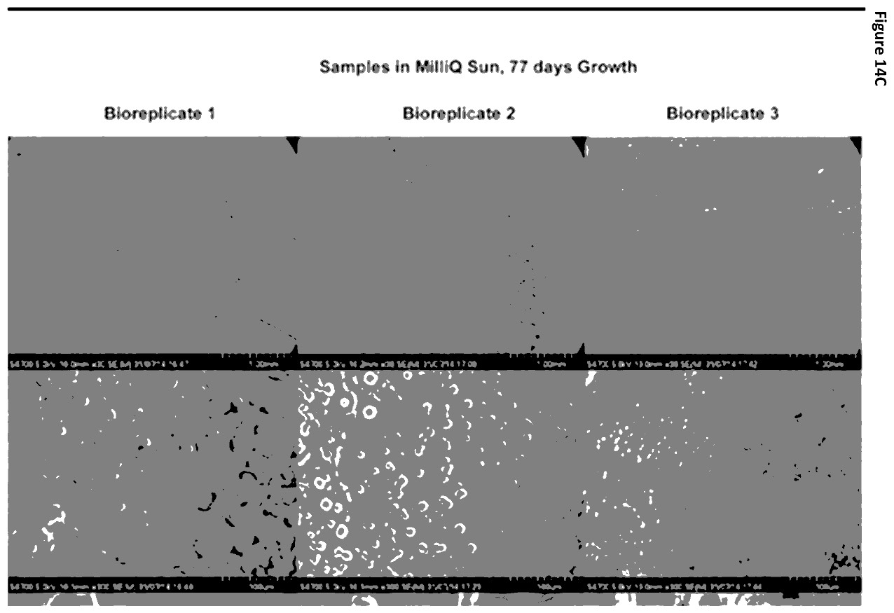

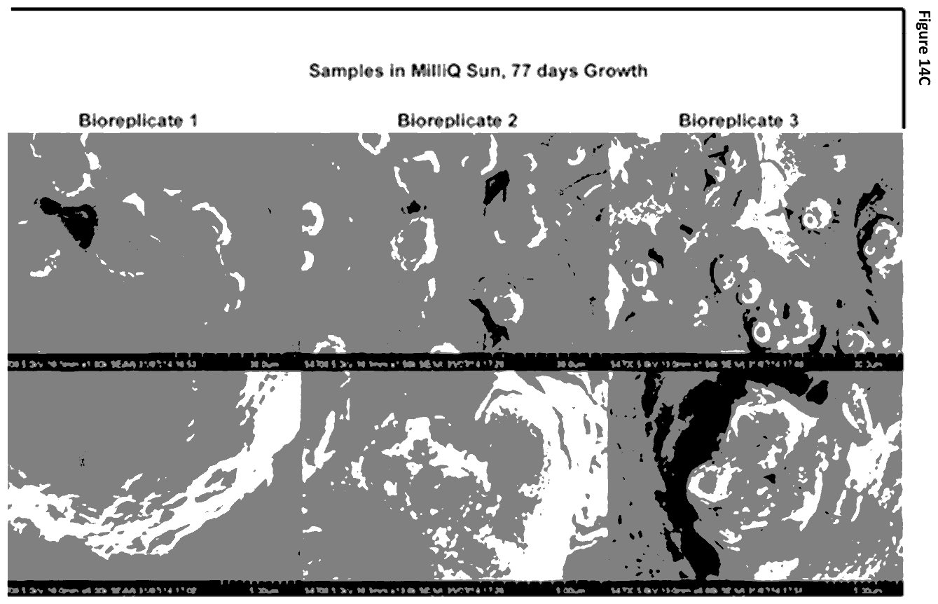

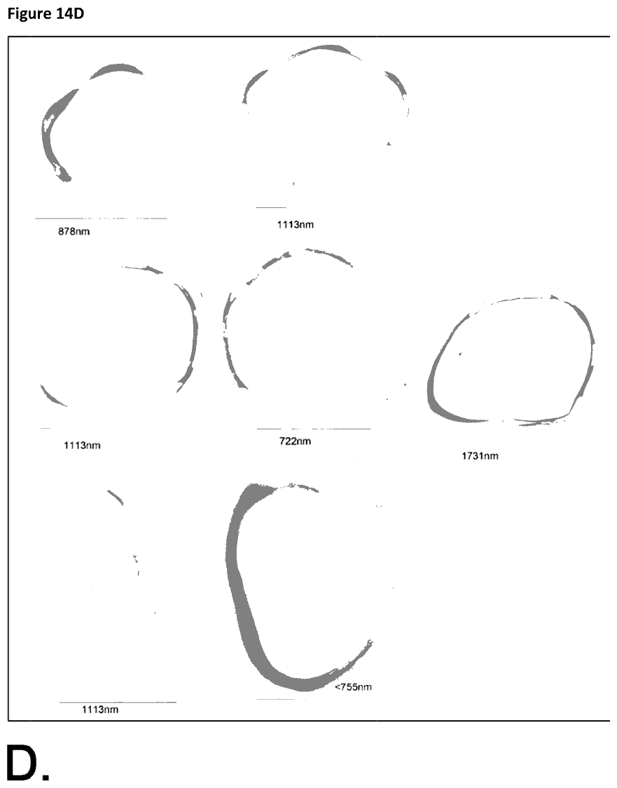

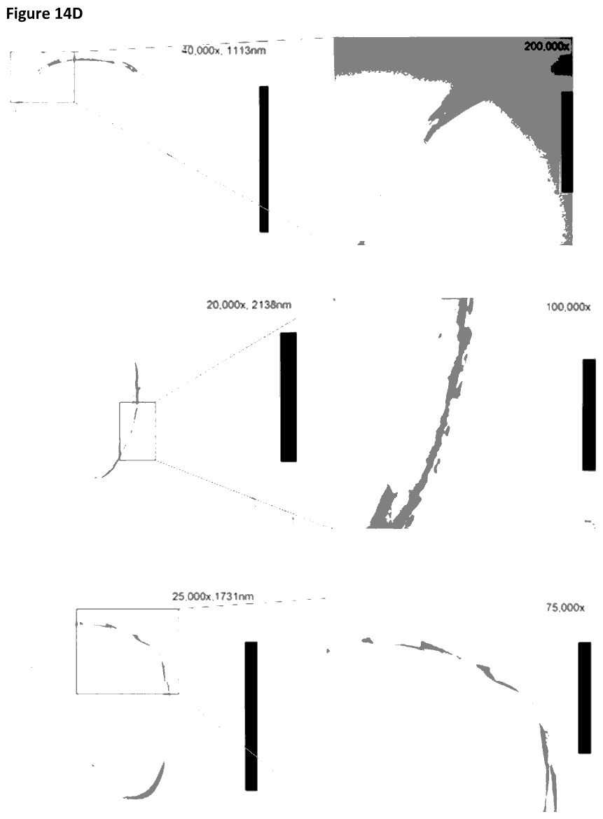

FIG. 14 illustrates electron microscopy images of the structures generated by protein synthesis according to the method. The relative scales can be seen (A.) between TEM and SEM, and the structures that are very similar in appearance to viral capsids (A right side panels). The structures were measured and counted using Omero software and the results are graphed in B. The PBS sun samples generate larger and less numerous structures, whilst the PBS Dark and MilliQ Sun generate numerous and much smaller structures. The reproducibility of the bio-replicates is quite remarkable and can be seen in panel C.

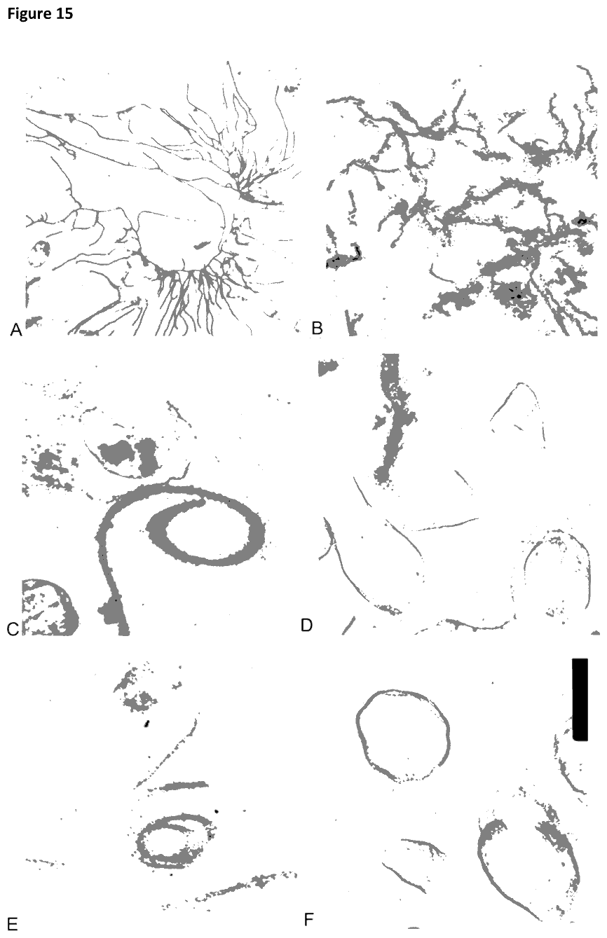

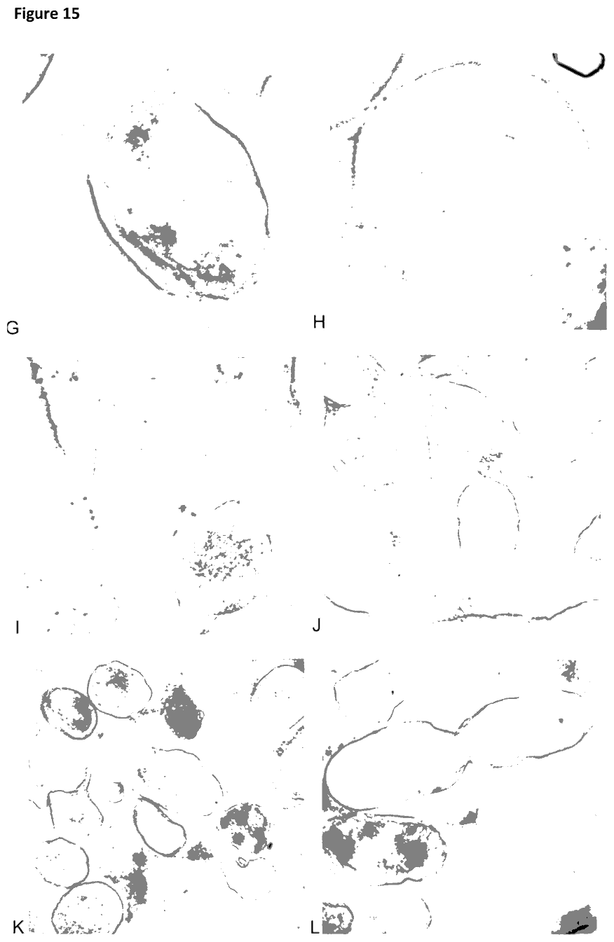

FIG. 15 illustrates ultra-microtomy was used to visualise the interior of the spheres. In earlier samples (A, B, C, ˜day 60) there is a prevalence of linear decorated strands of protein. In later samples (J, K, L, 678 days) the spherical structures are the majority of what can be seen. These vary from completely empty spheres (F) to slightly filled spheres, and very rarely densely filled structures. When compared to traditional cross sections of cells there is a distinct lack of structure and notable emptiness, none-the-less these are cell-like and could possibly represent prototype cell structures.

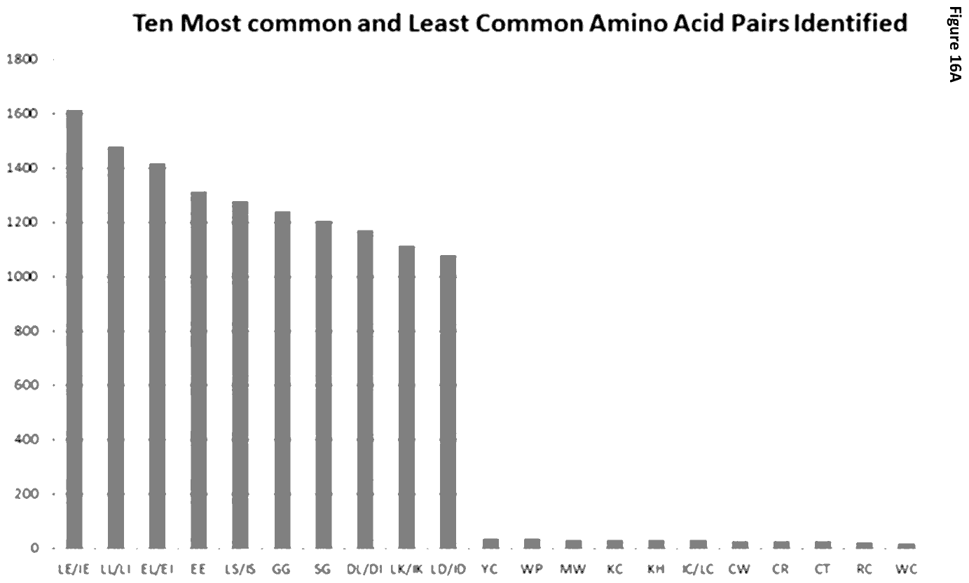

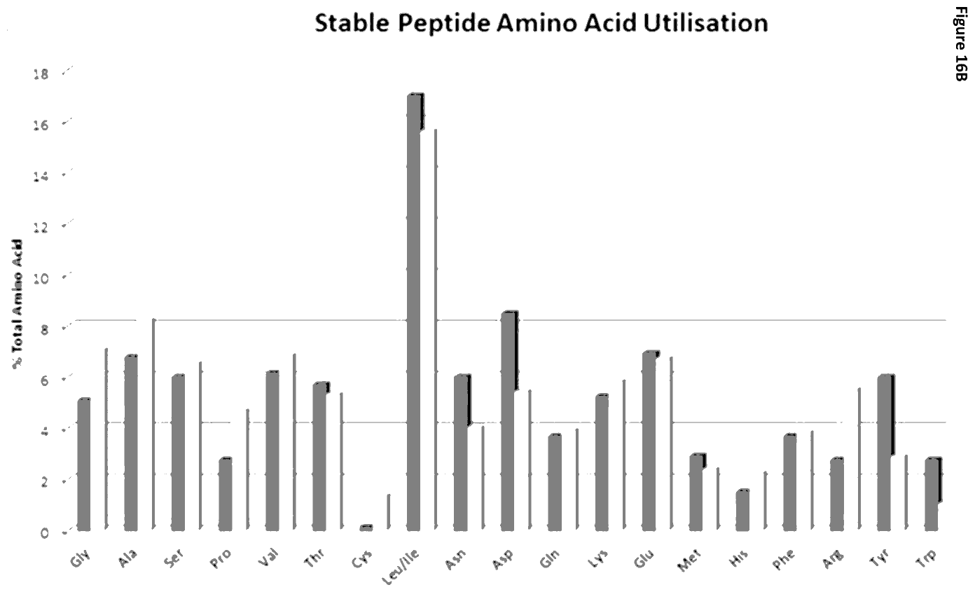

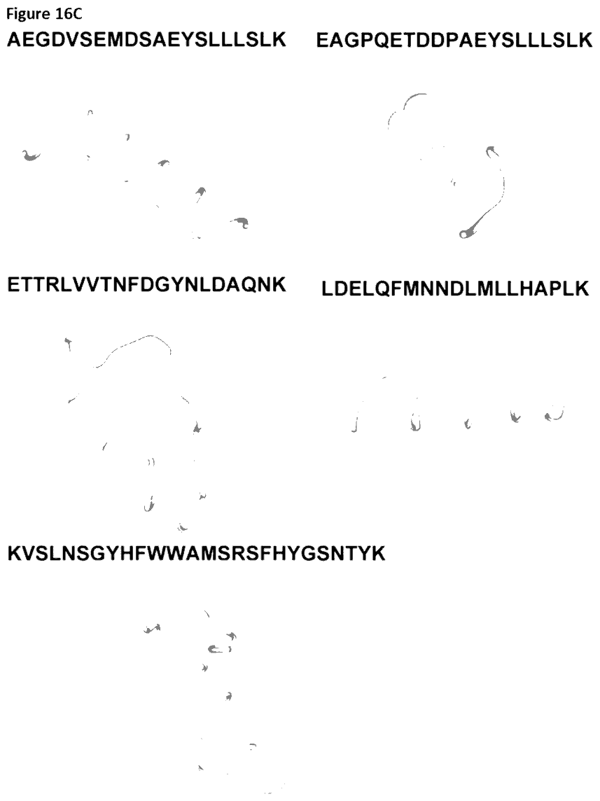

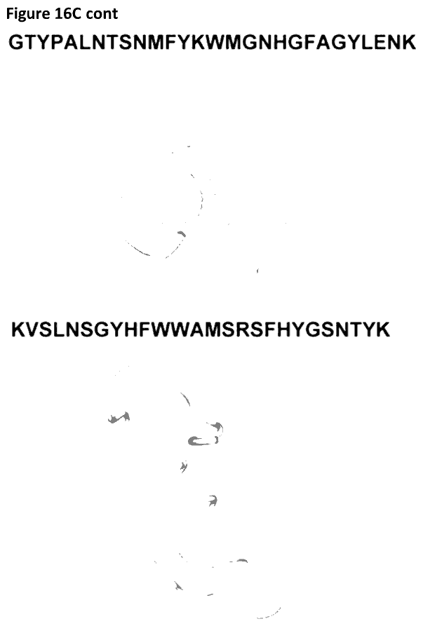

FIG. 16 illustrates A: Peptide pair analysis revealed the most common and least common amino acid pairings seen in all identified peptides. There appears to be a preference for non-polar amino acids (I, L, G) to pair with acidic amino acids (E, D). The least favourable pairings are between non polar and basic amino acids—and predictably the lowest abundance amino acids in the mix and B describes the utilisation of amino acids in the most stable peptides seen in all samples. It does not appear that there is a trend in the chemistry of amino acids (polar, non-polar and acidic amino acids all increased in usage). Aspartic acid and Asparagine are increased in usage but are thought to destabilize helices. C: Some examples of the most stable peptides. While random coil is present there is a tendency towards helical structures, suggesting the hydrogen bond formation between a backbone N—H group hydrogen to the backbone C═O group of the amino acid four residues earlier is sufficiently stabilizing in this context. Structures predicted by PEP-FOLD (http://bioserv.rpbs.univ-paris-diderot.fr/services/PEP-FOLD)

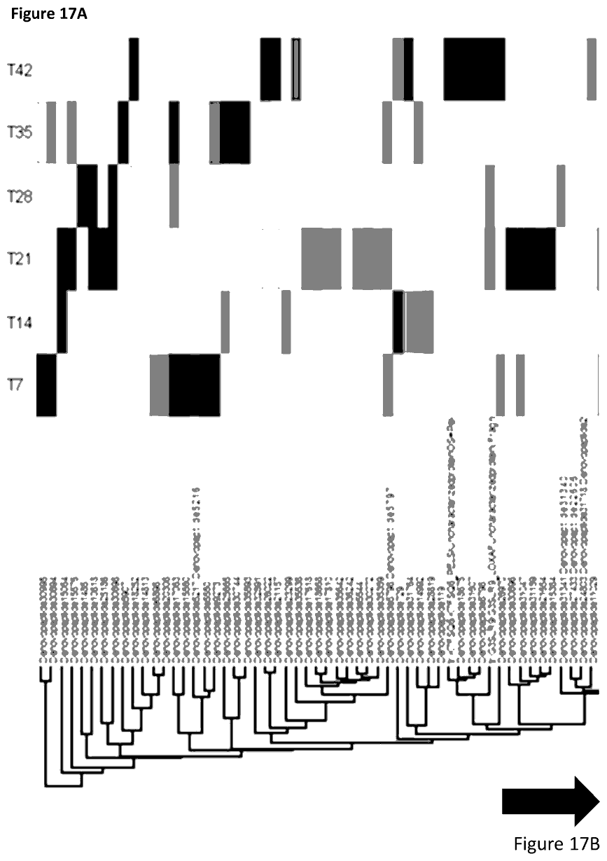

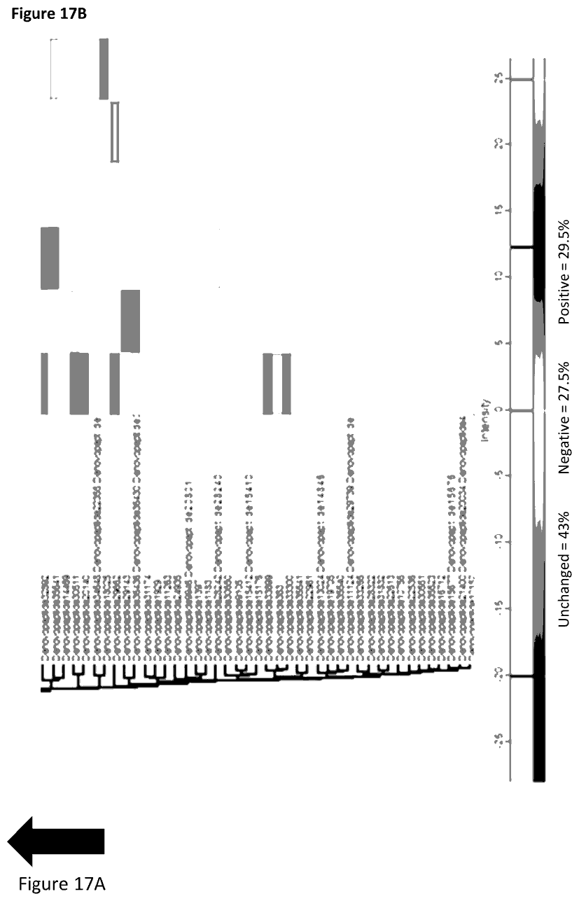

FIG. 17 illustrates comparison between constant heating of samples in the dark at 37° C. and the same sample set up but with a natural light source. The Log2 Intensity values of the standard set up proteins/peptides which showed increasing trends over the time course (indicating stable peptide generation and longevity) were subtracted from that of the 37° C. samples and the resulting values clustered.

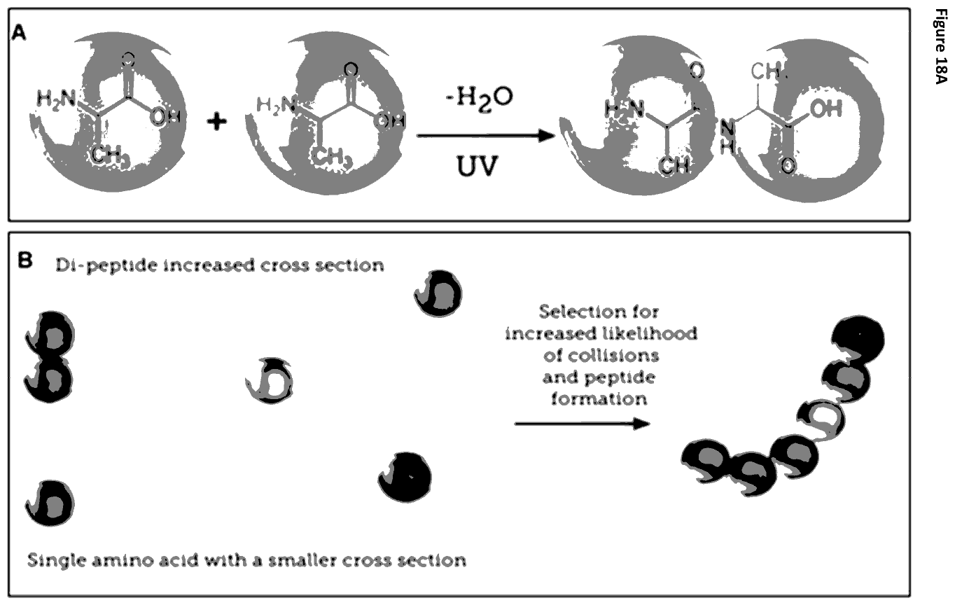

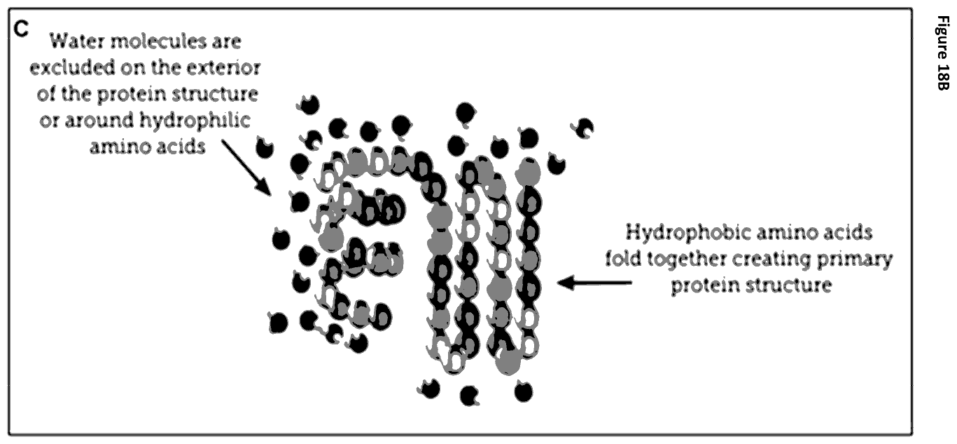

FIG. 18 illustrates a proposed method for the initial polymerisation of amino acids by the presence of amino acids in solution aided by energy (for example provided by sunlight energy) to overcome the required energy to form an amide bond wherein subsequent additions of amino acids to existing di-peptides are selected for in 2 ways (B). i) an enlarged cross-section increasing the likelihood of collisions with amino acids in the correct orientation, and ii) the energy required to add amino acids to existing di-peptides is 8× less than the initial amide bond. Additional local chemical environments favour of peptide/protein existence, inducing the folding of the peptide structures, excluding water molecules and forming secondary structures supported with hydrogen bonds and di-sulphide bonds (C). This generates structural stability and therefore longevity of peptides/proteins formed Reduction of entropy by forming spherical cell-like structures (D) to organize the proteins occurs. This again protects the structures formed, selecting in favour of these structures.

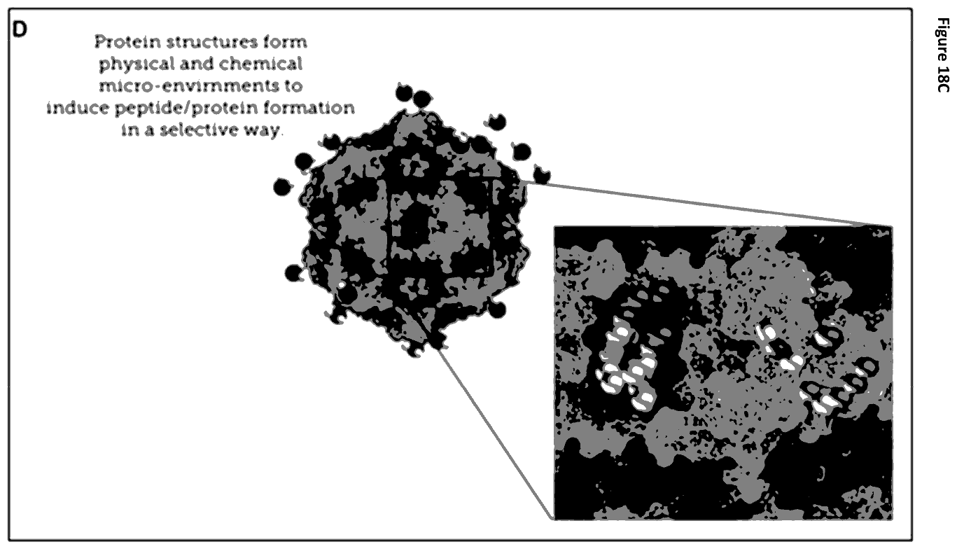

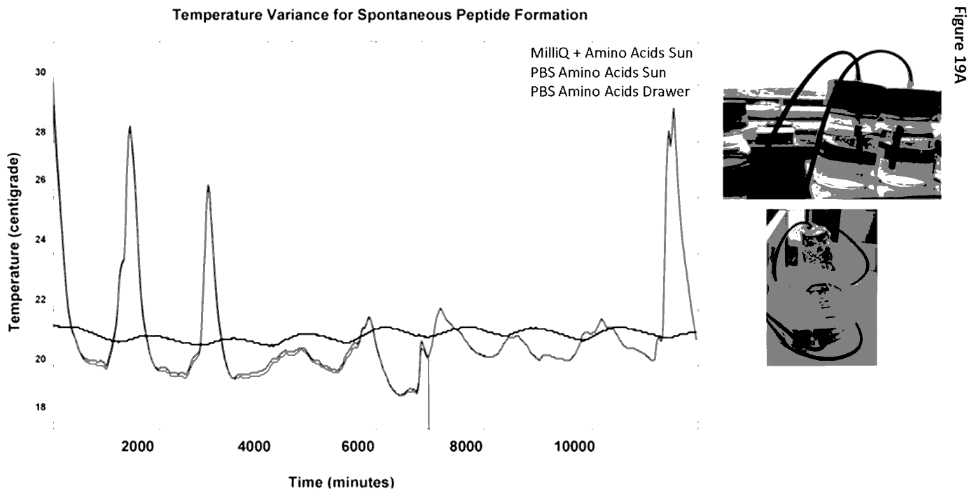

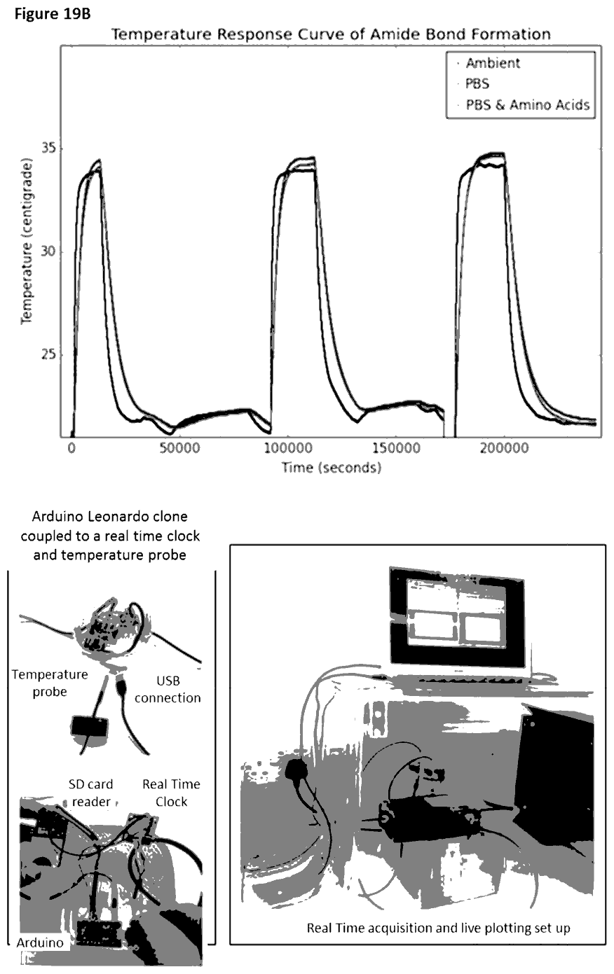

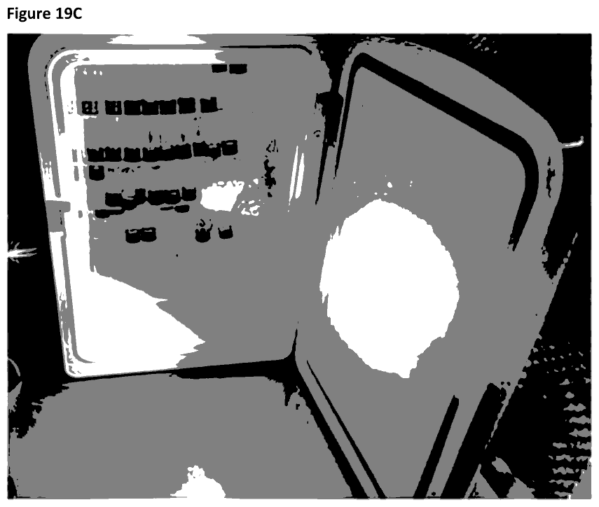

FIG. 19 illustrates apparatus to measure temperature fluctuations on the windowsill (A), then in a controlled heating environment without sunlight, to characterize the difference amino acids in solution made, and the amount of energy consumed in the apparatus (B) and finally the completely controlled environment used for all subsequent experiments (C). The system could be heated and cooled using a Peltier chip, with the addition of a sunlight replicating bulb (with a large range of wavelengths, including UV). Temperatures were monitored with medical grade temperature sensors. The apparatus was heated to 40° C. constantly for 12 hours with the bulb illuminated, and cooled to ambient temperature (˜18° C.) with the bulb off for 12 hours to replicate a controlled circadian cycle. Data presented here which was generated using the controlled environment was the sterilization experiment (1000 Gy irradiation samples). Temperatures were measured every 5 minutes with an accuracy of ±0.0625° C. Fluctuations in temperature on the window sills were 10.8125° C. (2096.7 J ° C.⁻¹g⁻¹), whilst the temperature fluctuations in the “dark” sample were 0.4444° C. (185.8 J ° C.⁻¹g⁻¹). To more accurately discern a difference between energy that may be consumed in the dehydration reactions required to make peptide bonds we used a controlled temperature environment and a control sample of PBS, and the PBS+amino acids solutions along with an ambient temperature measurement. Measurements were taken every 5 seconds for highly accurate heating and cooling curve description with the difference in area under the curve determined as 28962.97° C.²s, or 170° C.s, with the area being less in the PBS amino acids sample. The maximum of the peak in the PBS+amino acids was always less that the control, suggesting energy was always consumed in this system compared to the control. The resulting energy utilised by the PBS+amino acids sample was 1.31 kcal, suggesting that in one cycle of heating from 24° C. to 34° C. and cooling more than 1 mol of di-peptide formation was possible. It was determined that biological replicates of each condition had remarkable correlation to each other (Pearson correlations of up to 0.96) after 7 days of “growth” but this correlation broadens at ˜126 days growth, but always remains positive. It is hypothesized to be due to larger structures forming (evident in the TEM and SEM, causing the efficiency of peptide replication to tail off (via steric hindrance), when hydrophobic areas induce folding, reducing accessibility to protein surfaces.

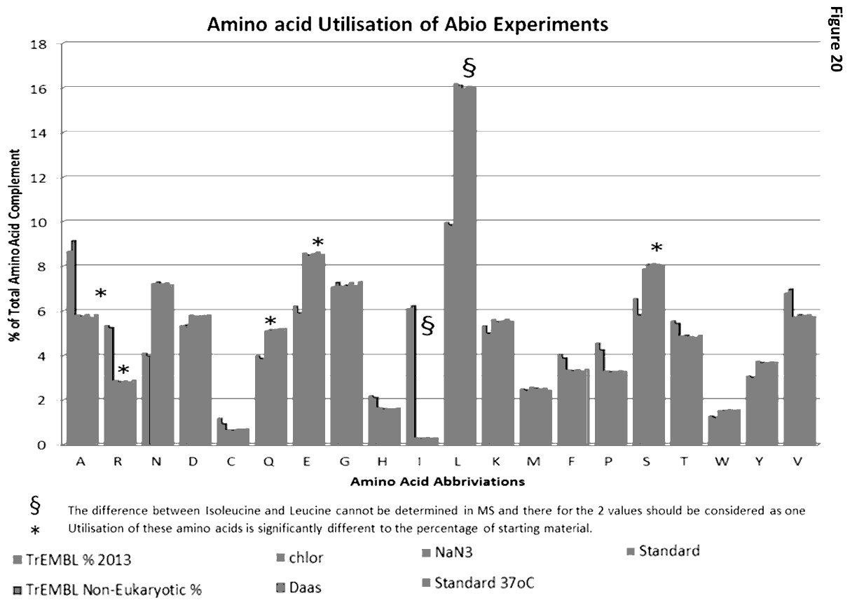

FIG. 20 illustrates Amino acid utilitisation in sample set up. The percentage of amino acids in TrEMBL % 2013 represents the percentages of amino acids in the starting solution. The differences in amino acids utilized compared to starting material are likely due to several factors such as trypsin digestion introducing bias (and therefore what we can measure in the Mass Spectrometer), chemical stability of amino acid sequence combinations and affinity to form amide bonds.

### Methods and Results

### Experiment 1

A synthesis experiment utilising heavy isotope labelled amino acids to distinguish the template peptide from the product peptide was carried out. This experiment was done with an internal standard (Benzoic acid) with chromatographic analysis (the industry standard for purity) and Mass Spectrometry to quantify the peptides (both light (template peptide) and heavy (newly synthesised) versions.

Eleven micrograms of synthetic peptide VR15 (VPDNLQQSLSDEAQR) SEQ ID NO: 1 was added to 1600 μl of amino acids solution at 0.2 g/ml concentration including Arginine which was 6 Daltons heavier than normal Arginine. Samples were incubated at 37° C., with a full spectrum light source, constantly for up to 4 hours.

Samples were taken at 2 and 4 hours of synthesis and run in triplicate on a reversed phase C18 chromatography column measuring UV absorbance at 216 nm (chromatograms in FIGS. 1B and 1C), and on the Mass Spectrometer in triplicate to measure the intensity of the heavy and light versions of the peptides (FIG. 1D).

### Results

The consistent increase in signal after 2 hours in the chromatogram of the synthesised peptide (see FIG. 1A) and the lack of any signal increase in the internal control (benzoic acid peak) shows clearly more VR15 was generated. The increase in signal was not due to instrument variation or any other external factor.

The SILAC quantification and labelling of newly synthesised peptides allows us to differentiate between the old and the new peptide, and this is also clear in the resulting peptide intensity measured with Mass Spectrometry.

### Experiment 2

During initial experiments it was found that proteins and peptides could be synthesised in the absence of cellular organelles and nucleic acids. Certain peptides were then selected to investigate a templated amplification process.

The following peptides were selected due to several factors
1. They do not occur in nature (with the exception of insulin).
2. They exhibited high copy numbers in initial experiments—suggesting they had been amplified. It was predicted that some peptides have unstable structure which is not conducive to longevity and replication. However, over 8000 different sequences were capable of being made reproducibly.
3. They had varying lengths to demonstrate the utility of the templated amplification process to a range of different peptides.

The peptide sequences initially investigated are set out in Table 1 below.

| Template | | | Concentration of | | | Concentration | |
|---|---|---|---|---|---|---|---|
| Peptide | Sequence | MW | template peptide | | Volume | of Amino acids | |
| MRFA | MRFA | 523.65 | 0.001 | g/ml | 10 ml | 0.01 | g/ml |
| SEQ ID | | | | | | | |
| NO: 4 | | | | | | | |
| MRFA* | MRFA | 523.65 | 0 | | 10 ml | 0.01 | g/ml |
| TR8 SEQ | TGASLNSR | 805.4163 | 0.00031 | g/ml | 10 ml | 0.01 | g/ml |
| ID NO: 5 | | | | | | | |
| VR9 | VMDSSYLSR | 1057.4983 | 0.00023 | g/ml | 10 ml | 0.063 | g/ml |
| SEQ ID | | | | | | | |
| NO: 2 | | | | | | | |
| Insulin | MALWMRLLP | 11974.026 | 0.00002 | g/ml | 100 ml | 0.2 | g/ml |
| SEQ ID | LLALLALWG | | | | | | |
| NO: 6 | PDPAAAFVN | | | | | | |
| | QHLCGSHLV | | | | | | |
| | EALYLVCGE | | | | | | |
| | RGFFYTPKT | | | | | | |
| | RREAEDLQV | | | | | | |
| | GQVELGGGP | | | | | | |
| | GAGSLQPLA | | | | | | |
| | LEGSLQKRG | | | | | | |
| | IVEQCCTSI | | | | | | |
| | CSLYQLENY | | | | | | |
| | CN | | | | | | |
| VR15 | RQAEDSLSQ | 1699.8246 | 0.00044 | g/ml | 10 ml | 0.01 | g/ml |
| | QLNDPV | | | | | | |

*A negative control experiment was set up wherein no template peptide was added to the solution.

The concentration of amino acids represents the total weight of amino acids per mL of solution. In each case, the relative amount of each amino acid present in the solution is proportional to the amount of the amino acid in the template peptide. By way of example, taking TR8, the composition of the amino acid mixture making up the 0.01 g/mL solution is approximately as follows T—0.0013 g/ml, G—0.0013 g/ml, A—0.0013 g/ml, S—0.0026 g/ml, L—0.0013 g/ml, N—0.0013 g/ml, and R—0.0013 g/ml (with no additional amino acids being present).

Table 1 shows a number of solutions used in the templated peptide synthesis process.

The solutions shown in Table 1 were prepared in a sterile environment (such as a laminar flow hood) and filtered through a 0.22 μm filter. The filtered solutions were placed in autoclaved schott bottles, sealed and then placed in an amplification apparatus.

The amplification apparatus subjected the samples to a controlled environment with a consistent temperature of 40° C. and constant source of full spectrum light. (Although it is possible to configure this apparatus in any number of ways).

Samples were taken (100 μl) daily under sterile conditions and analysed using MS (10 ml injected, 15 minute gradient, 2%-80% Acetonitrile 0.1% formic acid, Thermo Q Exactive Orbitrap) to determine the concentration of the peptides over time.

When a decrease in intensity was observed the amplification process was halted by placing the samples in a refrigerator. Absolute cessation of amplification is achieved by chromatographic separation of the peptides from the reagents (amino acids in solution).

The chromatographic separation of the product from reagents was achieved with a high flow rate large capacity C18 column, using reversed phase chromatography (the mobile phase used was A: 2% Acetonitrile, 0.1% Formic acid to B: 80% Acetonitrile, 0.1% Formic acid).

### Results

The amount of peptide produced was measured following purification and the results are indicated in Table 2 below.

| Peptide | Before (g) | After (g) | Amount made (g) | Increase (%) |
|---|---|---|---|---|
| Insulin | 0.0000342 | 0.000075959 | 0.000041759 | 122 |
| MRFA | 0.0085 | 0.010811 | 0.002310764 | 27 |
| VR9 | 0.0027 | 0.005286 | 0.00258607 | 96 |
| WK20* | 0.0003 | 0.000906 | 0.000605756 | 201 |

*WK20 SEQ ID NO: 3 (not shown in Table 1) is a synthetic peptide having the sequence WRWVLEHNVVEGNAVNLMFSK.

Table 2 shows the amount of template peptide before and after the amplification process.

In each case, the amount of template peptide increased following the amplification process. This is illustrated in FIG. 2.

It was observed that the shorter the peptide, the more rapid the amplification process. The drop in intensity thereafter is due to additional amino acids being added onto the product peptide, resulting in the loss of the mass of the desired/template peptide.

The mass spectral analysis for the synthesis of MRFA in the absence of a template (negative control, Table 1) is shown in FIG. 3, in comparison to the templated synthesis (carried out in the presence of an MRFA template). In contrast to the templated synthesis, no appreciable difference in the intensity of MRFA is observed in the peptide synthesis carried out in the absence of a template peptide.

### Experiment 3

### Excluding Other Causes of Peptide Amplification

To exclude the possibility of contamination (i.e. peptides present in starting material, carry over in the chromatography process, bacterial/viral contamination, or “life” of any sort) being present, the following experiments were carried out.
1. Analysis of samples for presence of DNA and RNA.
2. Use of D amino acids to amplify peptides (as most life forms cannot use these, or if they do they convert them into L amino acids).
3. Use of gamma irradiation to sterilise the samples and verify if amplification still occurs.
4. Perform the experiments in the presence of Chloramphenicol or Sodium Azide.
5. Testing the starting components for purity.

See FIG. 4.

### Methods

Commercial amino acids were measured out according to the percentages in Table 3 below to a total concentration of 1 g/100 ml. The amino acids were solubilised in sterile PBS (gibco 1× DPBS 14190-094). Solutions were mixed until complete solubilisation had occurred then passed through 0.22 μm stericup filters, and decanted 2 ml per autoclaved vial, in a cell culture fume hood with sterilised equipment. Vials were not opened again until an aliquot was required at the specified time point (again all done in a sterile environment). The samples were either analysed that day (if prior to day 14 incubation) or immediately reduced and alkylated, followed by in-solution digestion (with trypsin). The remainder of the sample was frozen at −80° C. An aliquot was taken for DNA and RNA analysis and analysed the day of collection also.

| Amino Acid | % | Amino Acid | % |
|---|---|---|---|
| Ala (A) | 8.73 | Gln (Q) | 4.00 |
| Arg (R) | 5.38 | Glu (E) | 6.20 |
| Asn (N) | 4.11 | Gly (G) | 7.10 |
| Asp (D) | 5.34 | His (H) | 2.18 |
| Cys (C) | 1.19 | Ile (I) | 6.09 |
| Leu (L) | 10.0 | Ser (S) | 6.52 |
| Lys (K) | 5.29 | Thr (T) | 5.52 |
| Met (M) | 2.50 | Trp (W) | 1.29 |
| Phe (F) | 4.03 | Tyr (Y) | 3.06 |
| Pro (P) | 4.55 | Val (V) | 6.81 |

Table 3 shows the total percentage of each of the 20 amino acids commonly found in the UniProtKB/TrEMBL protein database release October 2013 (http://www.ebi.ac.uk/uniprot/TrEMBLstats).

### DNA and RNA Detection

The Qubit commercial reagents were used according to the instructions for Qubit® dsDNA HS Assay Kit, Life Technologies, Q32851, and Qubit® RNA HS Assay Kit, Life Technologies, Q32855. Twenty microliters of sample were used for the assays. The samples were measured on the Qubit® 2.0 Fluorometer, Q32866.

### Marfey's Analysis of D Amino Acids Composition

One hundred microliters of each bio replicate of the D amino acids samples (T28=day 28) and the standard samples (T28=day 28) were pooled together resulting in 1 sample for each condition. The samples were rotary evaporated to dryness. 1 mL of 6M HCl was then added to the samples and they were heated at 155° C. for 80 minutes. The samples were then rotary evaporated to dryness. Samples were re-constituted with 300 μl of MilliQ water, and pH adjusted (to >5) with 3 μl of 10M NaOH. One hundred microliters were combined with 200 μl of Marfey's reagent (10 mg/ml in acetone) and 40 μl of 1M ammonium bicarbonate and gently shaken at 40° C. for 1 hour. The reaction was quenched with 20 μl of 2M HCl.

One hundred microliters of each sample were injected onto an XBridge® BEH 130 C18 Column, 130Å, 3.5 μm, 4.6 mm×250 mm. A gradient from 20% to 65% B over 45 minutes (A: 0.1% Formic acid, B: 80% Acetonitrile 0.1% formic acid) was used to separate the amino acids and the UV absorbance measured at 340 nm.

Mass Acquisition was performed using a Thermo Orbitrap QEaxactive, top 10 ions selected for ms/ms with an ms resolution of 140,000, ms/ms resolution 17,500, scanning from 150 m/z-2000 m/z.

### Gamma Irradiation

Samples were prepared as described above (solubilisation of amino acids, filtered and sterile) but with VR15 as a template peptide, and only with the required amino acids to constitute that peptide. Samples were prepared in triplicate, as well as vials containing live E. coli, as a positive control for sterilisation (irradiation with 1000 Gy, in sealed plastic bags). The positive controls were then spread on agar plates to verify sterilisation had occurred, and the resulting peptides in all samples analysed by mass spectrometry (as described above).

### Results

### DNA and RNA Detection

With reference to FIG. 5, in the process of testing for RNA and DNA presence in the samples, RNA was never detected (lower limit of detection <20 ng/ml). Samples subjected to full spectrum light source however, changed colour over time, and apparently generated a positive reading for DNA.

To determine whether this was truly DNA, benzonase (an enzyme known to degrade DNA) was added to the samples, which were then incubated for an hour. A positive control with benzonase (with known bacterial contamination) was used. The DNA intensity was roughly halved in the positive control while in the peptide synthesis samples the DNA intensity did not change. Therefore it was concluded that certain of peptides/proteins formed in the method, autofluoresce at the same wavelength as DNA (485/530 nm).

### D amino acid analysis

Most amino acids used in nature are the L chiral forms of amino acids, and most organisms cannot use them in standard protein production (they are however found commonly in peptidoglycan proteins in bacterial cell walls). The L forms of methionine, serine, alanine and tyrosine were substituted with the D versions in the normal amino acid mixture described above but otherwise the same experimental conditions were used.

The samples were then tested to test if the D amino acids had been converted into L amino acids by isomerases (e.g. such isomerases would be expected to be present if contaminant bacteria/life were present in the sample). A reagent known as Marfey's reagent (Nα-(2,4-Dinitro-5-fluorophenyl)-L-alaninamide) was utilised. On reaction with D amino acids, Marfey's reagent changes the retention time of those amino acids (when separated using chromatography) when compared to the L versions of the same amino acids.

The chromatograms illustrated in FIG. 6 exhibit this shift in retention time, from samples after 28 days of synthesis. These data further indicate there was no bacterial cellular contamination present in the samples.

### Gamma Irradiation

Synthetic peptide synthesis experiments were run in triplicate, but with 1000 Gy of radiation applied to the samples, which were then sealed to maintain the sterilisation achieved by the radiation. Positive controls of E. coli culture were included to verify the sterilisation occurred. If the peptide synthesis still occurred, then it was indicative that the process is not a result of contamination in the form of life in the samples.

A positive control for sterilization was included in the form of an E. coli culture. The non-irradiated samples (FIG. 7, A left side) show numerous colonies, and the irradiated samples (FIG. 7, A right side) show after 1000 Gy irradiation no growth at all, demonstrating effective sterilisation.

FIG. 7B is a histogram describing the peptide intensity distributions. The dark green population is the starting material, which has a low peptide count (as not many peptides had been generated) as well as lower median intensity. The standard incubated samples generated more peptides of higher intensity (i.e. more copies of peptides), with the irradiated samples showing a similar increase in peptide generation to that of the non-irradiated sample.

FIG. 7C is a Venn diagram showing the co-occurrence of peptides generated between conditions. The stochastic generation of peptides between the irradiated and not irradiated samples are equivalent.

The lack of distinct difference between these two sample sets suggests that the peptide synthesis is a chemical process not influenced by external living contamination in the samples.

### Chloramphenicol and Sodium Azide Containing Samples

The chloramphenicol and sodium azide samples were run in conjunction with the standard samples set up (sterile vial and amino acids solubilised in PBS) to allow a direct comparison to the abiotic peptide synthesis previously observed. The peptides were analysed in the same way, and compared to each other for any significant differences.

The Venn diagram shown in FIG. 8 illustrates the majority of peptides seen are identical in all samples. Additionally, there was no significant deficit in peptide production in the samples containing bacteriosides, which if the synthesis was a result of any bacterial or viral contamination, would have been expected to be reduced. This result further demonstrates that the described peptide synthesis process is a chemical one.

### Testing Starting Materials for Contamination

All commercial amino acids, solvents and digestive enzymes (trypsin) were analysed independently by mass spectrometry, and put through the de novo peptide identification software to determine if the peptides were present to begin with.

In all cases, there were no unexpected masses observed that were greater than the stated reagent.

### Example in Relation to Spontaneous Peptide Formation

Considering the hypothesis “Spontaneous peptide formation requires Sunlight and Phosphate”. Samples were set up in 3 different conditions (FIG. 12A) based on 2 assumptions. The first assumption was that Phosphate may be required given the energy unit used by most life is ATP—and the transfer of single phosphate molecules; therefore a simple source of phosphate in the form of Phosphate Buffered Saline was used. This had the additional properties of being a loose analogue to sea water (the environment where life may have begun 4 billion years ago in the Archaean). The second assumption was that energy from the sun was required to input sufficient kilojoules to overcome the amide bond formation step. Thus the samples were as follows: PBS Sun, PBS Dark, MilliQ Sun. The sample groups were intended as ‘PBS Sun’ being the proven positive, the ‘MilliQ Sun’ being a negative control and ‘PBS Dark’ another negative control.

However, it was determined that protein formation was apparent in all conditions. Thus, the inventor has determined that where there are building blocks for life (amino acids), in solution, with even minimal temperature fluctuations, spontaneous, cell and nucleic acid independent, protein synthesis will occur (FIG. 13).

### Example—Test of the Hypothesis “Constant Heating (37° C.) Will be Better for Spontaneous Peptide Generation than the Cycling Heat of a Day

Peptides/protein synthesis was compared which occurred in both standard (samples provided at room temperature on a window sill) and 37° C. samples, which showed increasing trends over the time course (indicating stable peptide generation and longevity). The data showed it is favourable to have sun light (variable wavelengths) and cycling temperature over constant temperature (and no sun light FIG. 17). The effect is likely greater than the change seen here due to the fact that the energy input of 37° C. was 24-7, whereas the energy and light input received on the window sill was at best half this.

### Conclusions

Detailed above is a method for peptide synthesis, based upon a previously unknown property of proteins and peptides acting as a template to replicate themselves, when placed in a solution of amino acids. Accompanying this data is extensive proof by several different means (gamma irradiation, D amino acids, bacteriocidal agents, as well as testing for presence of DNA and RNA) that this process is a chemical reaction and proceeds in the absence of nucleic acids, enzymes, co-enzymes, other cellular material and/or cells. Rather, the inventors believe that the templated peptide synthesis described herein may be driven by the provision of a low amount of energy (e.g. in the form of full spectrum light (including IR and UV) and/or gentle constant heat), the stoichiometry of the reagents and/or stereochemistry.

The spectra covered in FIGS. 9A-H are MALDI-TOF spectra verifying the purity of the starting commercially bought amino acids. The inventor has verified they were not containing anything aside from the specified amino acid (checking also for polymerisation of those amino acids into bigger peptides). This was indeed the case and the starting material was pure.

1.-24 (canceled)

25. A method of synthesising a peptide, the method comprising:
adding a quantity of a template peptide and amino acids capable of forming copies of the template peptide into an aqueous solution, and
providing at least 1.2 kcal/mol and light of wavelengths 350 to 700 nm and light intensity from 0 to 100% (in the form of Thermal IR, UV or full spectrum or mixture of any of these) to the solution in order to synthesise copies of the template peptide in solution.

26. The method according to claim 25, wherein the provision of energy comprises maintaining the solution at a constant temperature between about 10° C. and 100° C.

27. The method according to claim 25, wherein the provision of energy to the solution is cyclical.

28. The method according to claim 27, wherein the cyclical provision of energy comprises periodically increasing the heat of the solution and/or periodically exposing the solution to full spectrum light.

29. The method according to claim 25, wherein the solvent of the aqueous solution is pure or substantially pure water, or the aqueous solution comprises a phosphate buffered saline solution, acids or bases (such as HCl, Formic Acid, NaOH etc.).

30. The method according to claim 25, wherein the aqueous solution consists or consists essentially of pure or substantially pure water, the template peptide and the amino acids capable of forming copies of the template peptide.

31. The method according to claim 25, wherein the aqueous solution is sterile.

32. The method according to claim 25, wherein the aqueous solution comprises only those amino acids present in the template peptide.

33. The method according to claim 25, wherein the aqueous solution comprises the amino acids in a stoichiometric amount which equates to or is about equal to the stoichiometric amount of each amino acid found in the template peptide.

34. The method according to claim 25, wherein the total weight of all of the amino acids and the weight of the template peptide present in the aqueous solution are provided in a w/w (weight by weight) ratio of between 20,000 to 1 and 1 to 1, or between 10,000 to 1 and 10 to 1.

35. The method according to claim 25, wherein the total weight of the amino acids is provided in such an amount to provide a solution having a concentration between about 0.001 g/mL and 10 g/mL, or between about 0.005 g/mL and 5 g/mL, or between about 0.01 g/mL and 1 g/mL.

36. The method according to claim 25, wherein peptide synthesis is terminated after a period of between 10 minutes and 5 days.

37. The method according to claim 25, wherein peptide synthesis is terminated by removing the energy from the solution and/or by separation of the synthesised peptide from the aqueous solution.

38. The method according to claim 25, wherein the method is carried out at atmospheric pressure and/or in the presence of oxygen, or any other gas such as Nitrogen, hydrogen, CO₂ for example.

39. The method according to claim 25, wherein the peptide synthesis using a template peptide for amplification of itself takes place in the absence of nucleic acids, enzymes, co-enzymes, other cellular material and/or cells.


SEQUENCE LISTING (7 sequences)

SEQ ID NO: 1 - protein, 15 aa, Artificial Sequence
  VR15
VPDNLQQSLSDEAQR

SEQ ID NO: 2 - protein, 9 aa, Artificial Sequence
  VR9
VMDSSYLSR

SEQ ID NO: 3 - protein, 20 aa, Artificial Sequence
  WK20
WRWLEHNVVEGNAVNLMFSK

SEQ ID NO: 4 - protein, 4 aa, Artificial Sequence
  MRFA
MRFA

SEQ ID NO: 5 - protein, 8 aa, Artificial Sequence
  TR8
TGASLNSR

SEQ ID NO: 6 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 7 - protein, 15 aa, Artificial Sequence
  reverse VR15
RQAEDSLSQQLNDPV